(19) United States
(12) Patent Application Publication
(10) Pub. No.: US 2024/0336655 A1
(43) Pub. Date: Oct. 10, 2024

(54) AMPHIPHILIC PEPTIDES FOR NUCLEIC ACID AND PROTEIN DELIVERY

(71) Applicant: AJK BIOPHARMACEUTICAL, LLC, Irvine, CA (US)

(72) Inventors: Keykavous PARANG, Irvine, CA (US); Hamidreza Montazeri ALIABADI, Irvine, CA (US); Sandeep LOHAN, Lake Forest, CA (US); Dindyal MANDAL, Lake Forest, CA (US); Rakesh TIWARI, Irvine, CA (US); Eman MOHAMMED, Menoufia (EG); Ryley HALL, Tacoma, WA (US)

(21) Appl. No.: 18/294,623

(22) PCT Filed: Aug. 4, 2022
(86) PCT No.: PCT/US2022/039469

§ 371 Date: Feb. 2, 2024

Related U.S. Application Data
(60) Provisional application No. 63/229,149, filed on Aug. 4, 2021.

(51) Int. Cl. C07K 7/08; A01N 63/50; A01P 13/00; A61K 8/64; A61K 38/00; A61K 48/00; A61Q 19/00; C07K 7/64; C12N 15/87

(52) U.S. Cl. CPC C07K 7/08; A01N 63/50; A01P 13/00; A61K 8/64; A61K 48/0033; A61Q 19/00; C07K 7/64; C12N 15/87; A61K 38/00; A61K 2800/10

(57) ABSTRACT

Disclosed are transfecting agents for delivery of nucleic acids (e.g., DNA, plasmids, oligos, small interfering RNAs [siRNA], small hairpin RNA [shRNA], microRNAs [miRNA], Clustered Regularly Interspaced Short Palindromic Repeats [CRISPR]) or proteins (phosphoproteins, enzymes, antibodies, vaccines) using a peptide-based, pegylated peptide-based, a peptide attached to a lipophilic moiety (lipidated peptides), or lipophilic pegylated peptides to living cells.

### CROSS REFERENCE TO RELATED APPLICATIONS

This application claims the benefit of priority to U.S. Provisional Application No. 63/229,149, filed Aug. 4, 2021, the disclosure of which is incorporated by reference herein in its entirety.

### FIELD

The field of the invention is related to transfecting agents for delivery of nucleic acids (DNA, plasmids, oligos, small interfering RNAs [siRNA], small hairpin RNA [shRNA], microRNAs [miRNA], Clustered Regularly Interspaced Short Palindromic Repeats [CRISPR]) or proteins (phosphoproteins, enzymes, antibodies, vaccines) using a peptide-based, pegylated peptide-based, a peptide attached to a lipophilic moiety (lipidated peptides), or lipophilic pegylated peptides to living cells.

### BACKGROUND

The background description includes information that may be useful in understanding the present invention. It is not an admission that any of the information provided herein is prior art or relevant to the presently claimed invention, or that any publication specifically or implicitly referenced is prior art.

Most cellular functions and characteristics are controlled by the expression of different proteins (e.g., metabolic enzymes, transcription factors, and cell cycle proteins). The suppression of protein expression (also known as silencing) is performed by delivering double-stranded siRNAs, shRNA, miRNA, mRNA, or CRISPR/Cas9. RNA interference (RNAi) is an approach to suppress (or stop) the expression of a protein temporarily or permanently.

In 1998, Fire et al. demonstrated that the introduction of an exogenous RNA into a cell could interfere with the expression of a protein at mRNA level and confirmed that a double-stranded RNA was more effective for this purpose (Fire A, Xu S, Montgomery M K, Kostas S A, Driver S E, Mello C C. Potent and specific genetic interference by double-stranded RNA in Caenorhabditis elegans. Nature 1998; 391 (6669):806-11. Epub Mar. 5, 1998. doi: 10.1038/35888. PubMed PMID: 9486653). This breakthrough is considered by many the birth of RNA interference (RNAi) and the small interfering RNA (siRNA) approach. In the first decade of the 21^{st} century, siRNA was established as a reliable investigational tool in basic and translational research with the ability to target virtually any protein in mammalian cells and temporarily “silence” the expression of the target. Although siRNA silencing is temporary (unlike other RNAi approaches, e.g., short hairpin RNA or shRNA and micro RNA or miRNA), there are distinct advantages that explain its popularity in the field. Interestingly, siRNA is quite specific (unless unusually high concentrations are used) and does not require gene integration, further processing, or nucleus penetration. In addition, siRNA delivery resembles to that of a small molecule “drug” due to lack of transfection and insertion into the chromosome, which eliminates the need to isolate “transfected” cells. Protein silencing has not only played a crucial role in basic research and enhancing our understanding of protein functions (e.g., identification of regulators of cilliogenesis and ciliopathy genes (Wheway G, Schmidts M, Mans D A, Szymanska K, Nguyen T T, Racher H, Phelps I G, Toedt G, Kennedy J, Wunderlich K A, Sorusch N, Abdelhamed Z A, Natarajan S, Herridge W, van Reeuwijk J, Horn N, Boldt K, Parry D A, Letteboer S J F, Roosing S, Adams M, Bell S M, Bond J, Higgins J, Morrison E E, Tomlinson D C, Slaats G G, van Dam T J P, Huang L, Kessler K, Giessl A, Logan C V, Boyle E A, Shendure J, Anazi S, Aldahmesh M, Al Hazzaa S, Hegele R A, Ober C, Frosk P, Mhanni A A, Chodirker B N, Chudley A E, Lamont R, Bernier F P, Beaulieu C L, Gordon P, Pon R T, Donahue C, Barkovich A J, Wolf L, Toomes C, Thiel C T, Boycott K M, McKibbin M, Inglehearn C F, Consortium UK, University of Washington Center for Mendelian G, Stewart F, Omran H, Huynen M A, Sergouniotis P I, Alkuraya F S, Parboosingh J S, Innes A M, Willoughby C E, Giles R H, Webster A R, Ueffing M, Blacque O, Gleeson J G, Wolfrum U, Beales P L, Gibson T, Doherty D, Mitchison H M, Roepman R, Johnson C A. An siRNA-based functional genomics screen for the identification of regulators of ciliogenesis and ciliopathy genes. Nat Cell Biol. 2015; 17(8):1074-87. Epub Jul. 15, 2015. doi: 10 1038/ncb3201. PubMed PMID: 26167768; PMCID: PMC4536769), or identification of septins as coordinators of store-operated calcium entry (Sharma S, Quintana A, Findlay G M, Mettlen M, Baust B, Jain M, Nilsson R, Rao A, Hogan P G. An siRNA screen for NFAT activation identifies septins as coordinators of store-operated Ca²⁺ entry. Nature 2013; 499(7457):238-42. Epub Jun. 25, 2013. doi: 10.1038/nature12229. PubMed PMID: 23792561; PMCID: PMC3846693) and cancer progression), it has also led to significant medical findings such as treatment of Ebola virus-infected non-human primates (Thi E P, Mire C E, Lee A C, Geisbert J B, Zhou J Z, Agans K N, Snead N M, Deer D J, Barnard T R, Fenton K A, MacLachlan I, Geisbert T W. Lipid nanoparticle siRNA treatment of Ebola-virus-Makona-infected nonhuman primates. Nature 2015; 521(7552):362-5. Epub Apr. 23, 2015. doi: 10.1038/nature14442. PubMed PMID: 25901685; PMCID: PMC4467030) or inhibiting respiratory viral infections by nasally administered siRNA (Bitko V, Musiyenko A, Shulyayeva O, Barik S. Inhibition of respiratory viruses by nasally administered siRNA. Nat Med. 2005; 11(1):50-5. Epub Dec. 28, 2004. doi: 10.1038/nm1164. PubMed PMID: 15619632), which is especially relevant to the current coronavirus pandemic.

The double-stranded siRNA contains a passenger and a guide strand. The guide strand is incorporated into the RNA-induced silencing complex (RISC), while the passenger strand is degraded. The guide strand acts as a complementary sequence to the messenger RNA, and therefore, binds to the targeted mRNA, which triggers the Argonaute 2 (an essential catalytic protein in RISC) to cleavage the mRNA into small pieces, which will be degraded rapidly by RNases. This process is known as post-transcriptional gene silencing (Akinc et al., Targeted delivery of RNAi therapeutics with endogenous and exogenous ligand-based mechanisms. Mol Ther, 2010, 18(7):1357-64).

This complex intervenes in the degradation process of the siRNA sense strand and utilizes the preserved anti-sense strand to identify the complementary sequences of mRNA. Several delivery systems have been evaluated to improve the efficiency of siRNA, which include polymers, lipids, liposomes, and carbon nanotubes (Liu et al., SiRNA delivery systems based on neutral cross-linked dendrimers. Bioconjug Chem, 2012, 23(2):174-83; Chabot et al., Targeted electro-delivery of oligonucleotides for RNA interference: siRNA and antimiR. Adv Drug Deliv Rev, 2015, 81:161-8; de Fougerolles et. al., Interfering with disease: a progress report on siRNA-based therapeutics. Nat. Rev Drug Discov, 2007, 6(6):443-53; Golzio et al., In vivo gene silencing in solid tumors by targeted electrically mediated siRNA delivery. Gene Ther, 2007, 14(9):752-9; Oh et al., siRNA delivery systems for cancer treatment. Adv Drug Deliv Rev, 2009, 61(10):850-62). However, the majority of the delivery systems exhibited significant cytotoxicity and/or demonstrated limited in vivo and clinical potential. For example, poly (ethyleneimine) (PEI) is a commonly used polymeric carrier for siRNA delivery. The use of PEI facilitates the endosomal release of siRNA in the cellular cytoplasm. However, PEI induces toxicity contributes to low transfection efficiency (Guo et al., Engineering RNA for targeted siRNAdelivery and medical application. Adv Drug Deliv Rev, 2010, 62(6):650-66).

Cell-penetrating peptides (CPPs) have shown promising results in siRNA delivery (among other biomolecules as cargo). The repetitive nature of the molecular structure of peptides and the versatile characteristics of its building blocks (amino acids) offer structural flexibility that could be advantageous in optimizing these carriers for the delivery of different molecules (Aliabadi H M, Landry B, Sun C, Tang T, Uludag H. Supramolecular assemblies in functional siRNA delivery: where do we stand? Biomaterials 2012; 33(8):2546-69. doi: 10.1016/j.biomaterials.2011.11.079. PubMed PMID: 22209641). Cyclic peptides (CPs) have been studied increasingly in cancer and gastrointestinal disorders because of their enhanced enzymatic stability and cell specificity versus linear peptides (Katsara M, Tselios T, Deraos S, Deraos G, Matsoukas M T, Lazoura E, Matsoukas J, Apostolopoulos V. Round and round we go: cyclic peptides in disease. Curr Med Chem. 2006; 13(19):2221-32. PubMed PMID: 1691835 Nasrolahi Shirazi A, Mandal D, Tiwari R K, Guo L, Lu W, Parang K. Cyclic peptide-capped gold nanoparticles as drug delivery systems. Mol Pharm. 2013; 10(2):500-11. doi: 10.1021/mp300448k. PubMed PMID: 229984730; Jing X, Jin K. A gold mine for drug discovery: Strategies to develop cyclic peptides into therapies. Med Res Rev. 2019. Epub Oct. 11, 2019. doi: 10.1002/med.21639. PubMed PMID: 31599007). Application of naturally occurring amino acid-based CPs in drug delivery has become a subject of major interest. We recently reviewed cyclic CPPs as drug delivery tools (Park S E, Sajid M I, Parang K, Tiwari R K. Cyclic Cell-Penetrating Peptides as Efficient Intracellular Drug Delivery Tools. Mol Pharm. 2019; 16(9):3727-43. Epub Jul. 23, 2019. doi: 10.1021/acs.molpharmaceut.9b00633. PubMed PMID: 31329448) and have designed homochiral CPs containing tryptophan (W) and arginine (R) residues for enhanced delivery of lamivudine, phosphopeptides, doxorubicin (Mandal D, Nasrolahi Shirazi A, Parang K. Cell-penetrating homochiral cyclic peptides as nuclear-targeting molecular transporters. Angew Chem Int Ed Engl. 2011; 50(41):9633-7. doi: 10.1002/anie.201102572. PubMed PMID: 21919161; Nasrolahi Shirazi A, Tiwari R, Chhikara B S, Mandal D, Parang K. Design and biological evaluation of cell-penetrating peptide-doxorubicin conjugates as prodrugs. Mol Pharm. 2013; 10(2):488-99. doi: 10.1021/mp3004034. PubMed PMID: 23301519; Darwish S, Sadeghiani N, Fong S, Mozaffari S, Hamidi P, Withana T, Yang S, Tiwari R K, Parang K. Synthesis and antiproliferative activities of doxorubicin thiol conjugates and doxorubicin-SS-cyclic peptide. Eur J Med Chem. 2019; 161:594-606. doi: 10.1016/j.ejmech.2018.10.042. PubMed PMID: 30396106; Oh D, Darwish S A, Shirazi A N, Tiwari R K, Parang K. Amphiphilic bicyclic peptides as cellular delivery agents. Chem Med Chem. 2014; 9(11):2449-53. doi: 10.1002/cmdc.201402230. PubMed PMID: 25047914, paclitaxel, camptothecin El-Sayed N S, Shirazi A N, Sajid M I, Park S E, Parang K, Tiwari R K. Synthesis and Antiproliferative Activities of Conjugates of Paclitaxel and Camptothecin with a Cyclic Cell-Penetrating Peptide. Molecules 2019; 24(7). Epub Apr. 14, 2019. doi: 10.3390/molecules24071427. PubMed PMID: 30978971; PMCID: PMC6480016), curcumin (Shirazi A N, El-Sayed N S, Tiwari R K, Tavakoli K, Parang K. Cyclic Peptide Containing Hydrophobic and Positively Charged Residues as a Drug Delivery System for Curcumin. Curr Drug Deliv. 2016; 13(3):409-17. PubMed PMID: 26511089), selenium and gold nanoparticles (Nasrolahi Shirazi A, Tiwari R K, Oh D, Sullivan B, Kumar A, Beni Y A, Parang K. Cyclic peptide-selenium nanoparticles as drug transporters. Mol Pharm. 2014; 11(10):3631-41. doi: 10.1021/mp500364a. PubMed PMID: 25184366; PMCID. PMC4186687; Do H, Sharma M, El-Sayed N S, Mahdipoor P, Bousoik E, Parang K, Montazeri Aliabadi H. Difatty Acyl-conjugated linear and cyclic peptides for siRNA Delivery. ACS Omega 2017; 2:6939-57), oligodeoxynucleotides, and siRNA (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel) 2019; 11(4). doi: 10.3390/polym11040703. PubMed PMID: 30999603; PMCID: PMC6523661; Shirazi A N, Paquin K L, Howlett N G, Mandal D, Parang K. Cyclic peptide-capped gold nanoparticles for enhanced siRNA delivery. Molecules 2014; 19(9):13319-31. doi: 10.3390/molecules190913319. PubMed PMID: 25170952; PMCID: PMC6271229). Selected CPs have unique and intrinsic properties that offer several advantages over linear peptides due to their conformationally constrained structure, including nuclear delivery, endocytosis-independent uptake, serum stability, low cytotoxicity, biocompatibility, hydrophobic and anionic drug entrapment through non-covalent interactions, selective tumor targeting. CPs efficiency in siRNA delivery could be enhanced by proper balance between positive charge density and hydrophobicity of the structure, which has been reported by us (Mozaffari S, Bousoik E, Amirrad F, Lamboy R, Coyle M, Hall R. Alasmari A, Mahdipoor P, Parang K, Montazeri Aliabadi H. Amphiphilic Peptides for Efficient siRNA Delivery Polymers (Basel), 2019; 11(4). doi: 10.3390/polym11040703. PubMed PMID: 30999603; PMCID: PMC6523661, Do H, Sharma M, El-Sayed N S, Mahdipoor P, Bousoik E, Parang K, Montazeri Aliabadi H. Difatty Acyl-conjugated linear and cyclic peptides for siRNA Delivery. ACS Omega, 2017; 2:6939-57) and others (van Asbeck A H, Beyerle A, McNeill H, Bovee-Geurts P H, Lindberg S, Verdurmen W P, Hallbrink M, Langel U, Heidenreich O, Brock R. Molecular parameters of siRNA-cell penetrating peptide nanocomplexes for efficient cellular delivery. ACS Nano, 2013; 7(5):3797-807. Epub Apr. 23, 2013. doi: 10.1021/nn305754c. PubMed PMID: 23600610). However, there is no commercial transfecting agent with unique intrinsic properties of a cyclic peptide in the market. Therefore, the development of a peptide-based transfection agent with a non-toxic profile, stability in serum, and diverse nucleic acid delivery profile is a significant need. The compositions and methods disclosed herein address these and other needs.

Cyclic peptides containing alternate tryptophan and arginine residues [WR]₅ and [WR]₄ and the corresponding peptide-capped gold nanoparticles were found to act as efficient molecular transporters of siRNA in human cervix adenocarcinoma (HeLa) cells (Shirazi A N, Paquin K L, Howlett N G, Mandal D, Parang K. Cyclic peptide-capped gold nanoparticles for enhanced siRNA delivery. Molecules, 2014; 19(9):13319-31. doi: 10.3390/molecules190913319. PubMed PMID: 25170952; PMCID: PMC6271229). Flow cytometry studies showed that [WR]₅ and [WR]₅-capped gold nanoparticles improved the intracellular uptake of siRNA versus siRNA alone. It was also found that both delivery platforms were less toxic when compared with lipofectamine. Fluorescence microscopy data confirmed the localization of fluorescence-labeled siRNA in the presence of [WR]₅ and [WR]₅-capped gold nanoparticles. Thus, the positively charged residues on the peptide could have electrostatic interactions with negatively charged phosphate residues in the phospholipid bilayer and siRNA. Furthermore, the hydrophobic tryptophan groups could interact with hydrophobic residues in the lipid membrane. The efficacy of fatty acid-conjugated linear and cyclic arginine/lysine peptides in delivering siRNA to breast cancer cells and the efficacy of protein silencing via this approach was also shown (Do H, Sharma M, El-Sayed N S, Mahdipoor P, Bousoik E, Parang K, Montazeri Aliabadi H. Difatty Acyl-conjugated linear and cyclic peptides for siRNA Delivery. ACS Omega, 2017; 2:6939-57). A variety of linear and cyclic peptides were synthesized with various fatty acyl conjugations with different lengths (from C2 to C18) and degree of saturation. Most recently, the potential of [WR]₅ for siRNA delivery was further confirmed (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel), 2019; 11(4). doi: 10.3390/polym11040703. PubMed PMID: 30999603; PMCID: PMC6523661).

There remains a need for improved pharmaceutical compositions including small molecule drugs, nucleic acids, including DNA, plasmids, oligos, small interfering RNAs [siRNA], small hairpin RNA [shRNA], microRNAs [miRNA], and Clustered Regularly Interspaced Short Palindromic Repeats [CRISPR], and proteins, such as phosphoproteins, enzymes, antibodies, and vaccines. There remains a need for improved methods of delivering such compositions and active agents to target organs and cells within the body. There remains a need for improved compounds capable of transporting therapeutic compounds, including nucleic acids and proteins, across cell membranes. There remains a need for such compounds having improved transfection rates, reduced toxicity, increased stability, and/or improved pharmacokinetic properties.

### SUMMARY

Disclosed herein are compounds, compositions, methods for making and using such compounds and compositions. In specific aspects, disclosed are peptide-based systems containing hydrophobic amino acids (e.g., tryptophan), charged amino acids (e.g., arginine), lysine linker, and/or sulfur-containing amino acids (e.g., cysteine), which can be used as nucleic acid transfecting agents or protein delivery in living cells.

### BRIEF DESCRIPTION OF FIGURES

The accompanying Figures, which are incorporated in and constitute a part of this specification, illustrate several aspects and together with the description serve to explain the principles of the invention.

FIG. 1 shows the chemical structure of representative linear and cyclic peptides in Classes I-XI.

FIG. 2 shows the sequence of selected peptides in Classes XII-XV.

FIG. 3 shows the synthesis of lipidated and pegylated linear peptides.

FIG. 4 shows the synthesis of lipidated and pegylated linear peptides with beta-alanine as a spacer.

FIG. 5 shows the synthesis of lipidated cyclic peptides with Beta-alanine as a spacer.

FIG. 6 shows the synthesis of lipidated and pegylated linear peptides with PEG1 as a spacer.

FIG. 7 shows the synthesis of lipidated cyclic peptides with amino-PEG-1-acid as a spacer.

FIG. 8 shows the serum stability of siRNA complexed with C4, H4, H4-PEG550, H4-PEG2000, and CP-PEG2000 as compared to “naked” siRNA (FBS 25%, incubation time 24 h).

FIG. 9 is a graph showing cytotoxicity of PAB006 (a-f) in MDA-MB-231 breast cancer cells.

FIG. 10 shows cytotoxicity of peptide/siRNA complex in MDA-MB-231 cells.

FIG. 11 shows the cytotoxicity of C4 and H4 in MDA-MB-231 after 48 h incubation.

FIGS. 12A-12B show cytotoxicity of (12A) H4/H4-PEG mixtures and (12B) C4/C4-PEG mixture in MDA-MB-231 cells after 48 h incubation (N/P ration: 40).

FIG. 13 shows cellular uptake of Alexa 488 labeled siRNA in the presence of peptides in MDA-MB-231 cells.

FIG. 14 is a group of confocal microscopy micrographs showing the cellular uptake of siRNA/peptide complexes at N/P ratio of 20 compared to Lipofectamine™2000 (Lipo.).

FIG. 15 shows gel retardation assay of siRNA condensed with peptide H4, C4, H4-PEG550, H4-PEG200, C4-PEG2000 at different N/P ratios.

FIG. 16 shows Western Blot used to analyze STAT3 silencing using PAB006 derivatives.

FIG. 17 shows STAT3 siRNA transfection using PAB006 (Lower concentration of peptide/siRNA).

FIG. 18 shows the comparison of STAT3 protein silencing activities of C4 and H4 in MDA-MB-231 cells.

FIG. 19 shows STAT 3 silencing using H4/Peptide-PEG mixture (cell line: MDA-MB-231, incubation time (48 h), N/P 40, siRNA 100 nM).

FIG. 20 shows STAT 3 silencing using C4/Peptide-PEG mixture (MDA-MB-231, Incubation time: 48 h, N/P 40, siRNA 100 nM).

FIG. 21 shows STAT3 silencing using peptide/peptide-PEG mixture at lower concentrations of siRNA (50 nM) (MDA-MB-231, N/P 40, siRNA 50 nM, lipofectamine 100 nM).

FIG. 22 shows PAB101 as a transfection agent for CRISPR/CAS9.

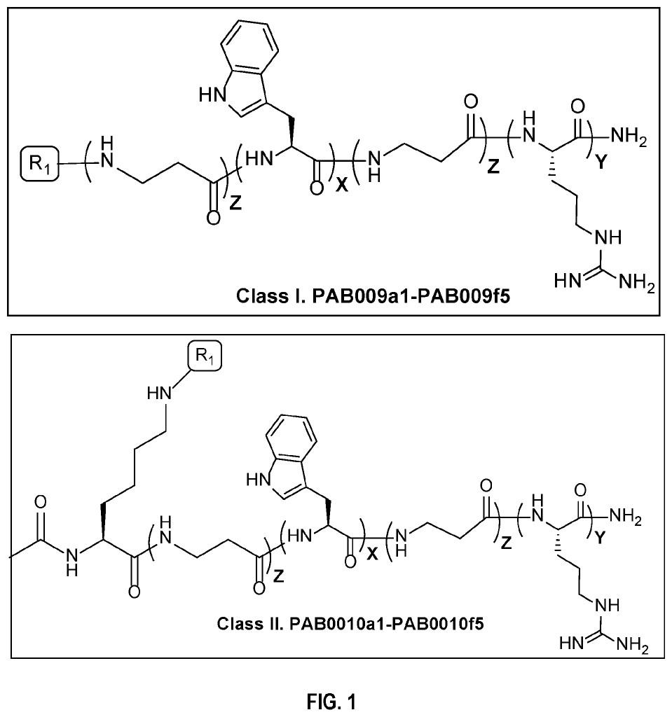

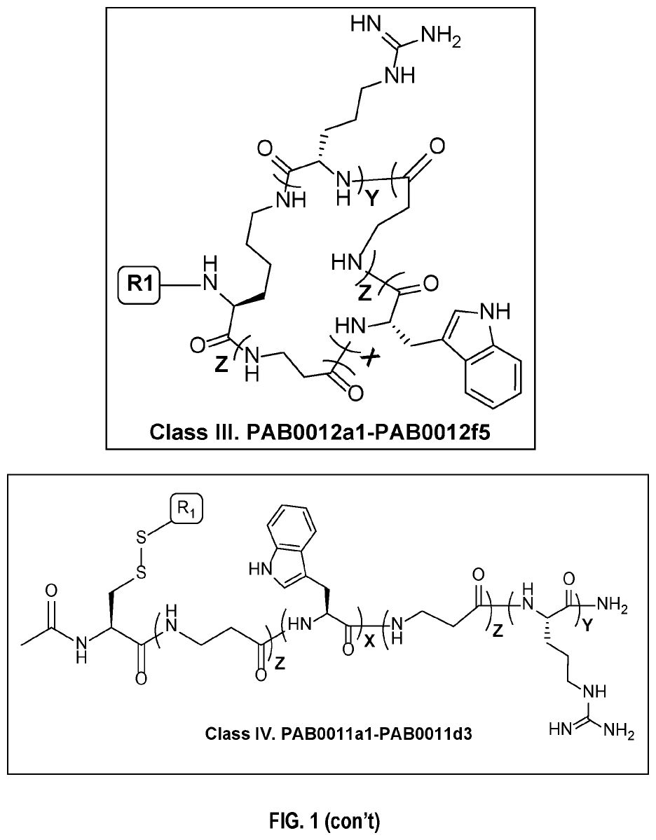

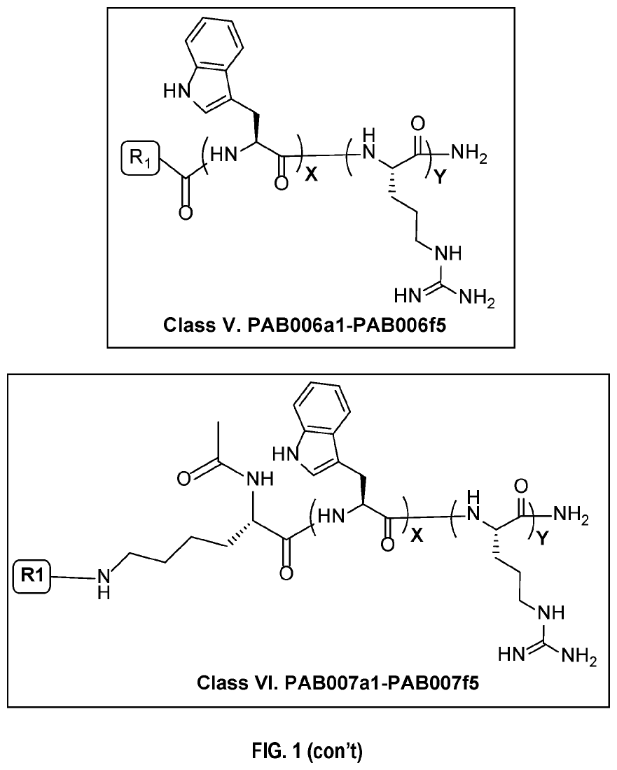

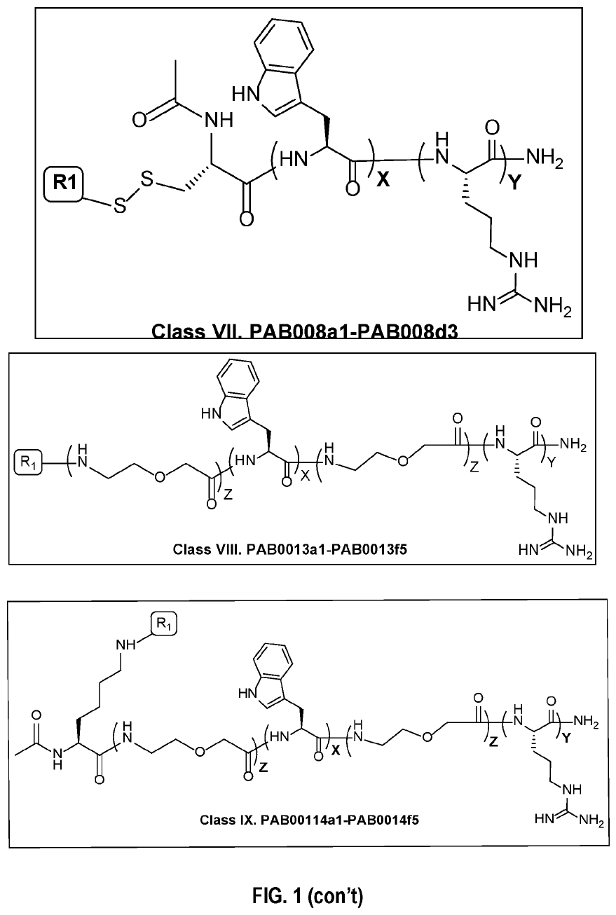

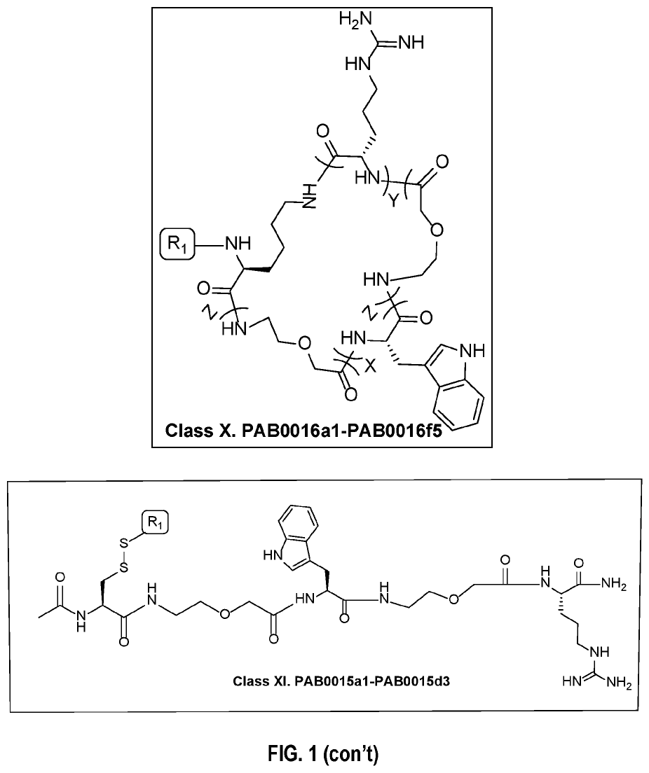

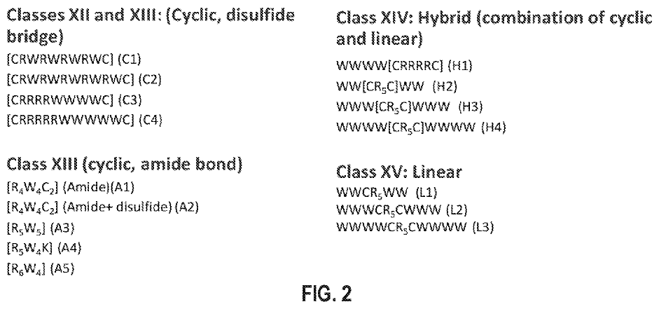

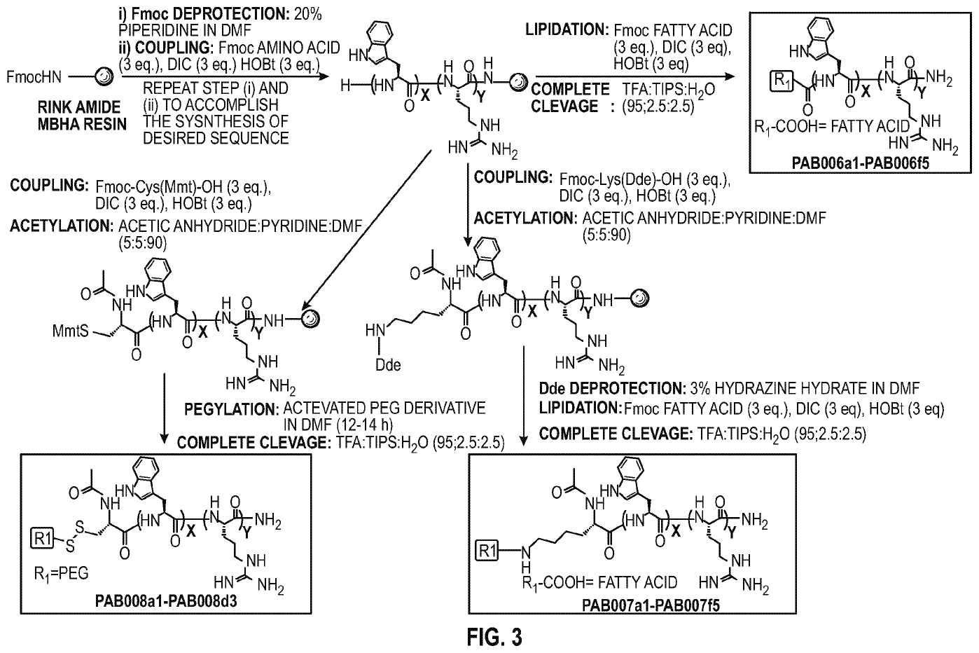

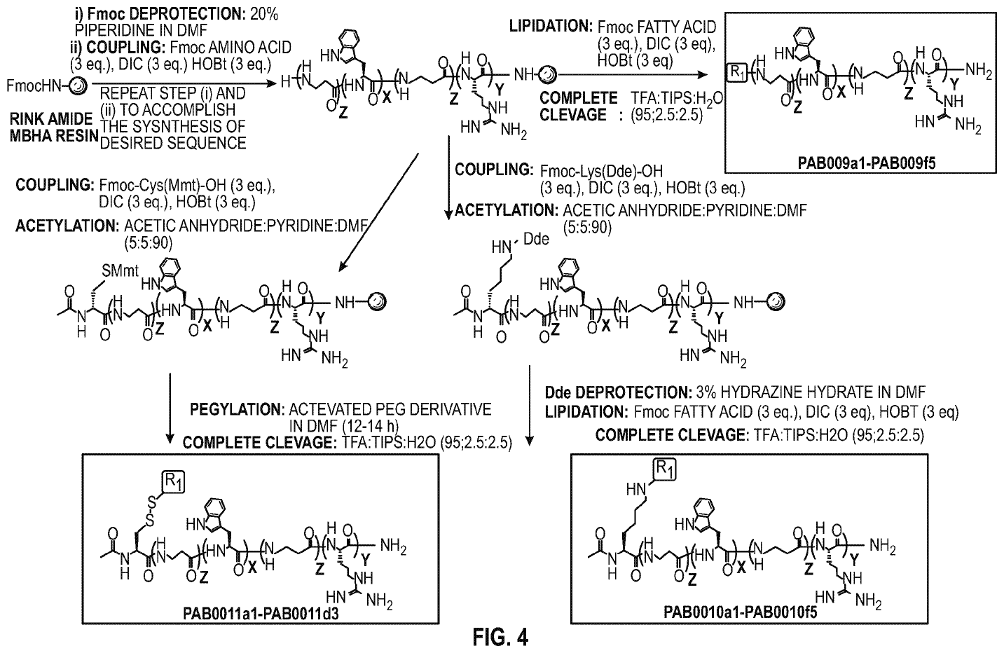

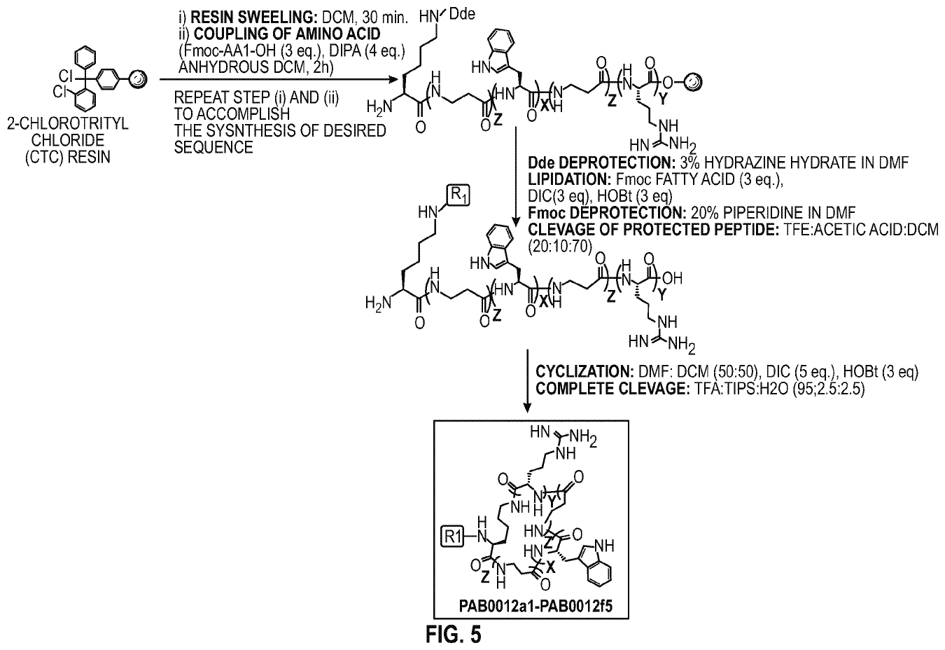

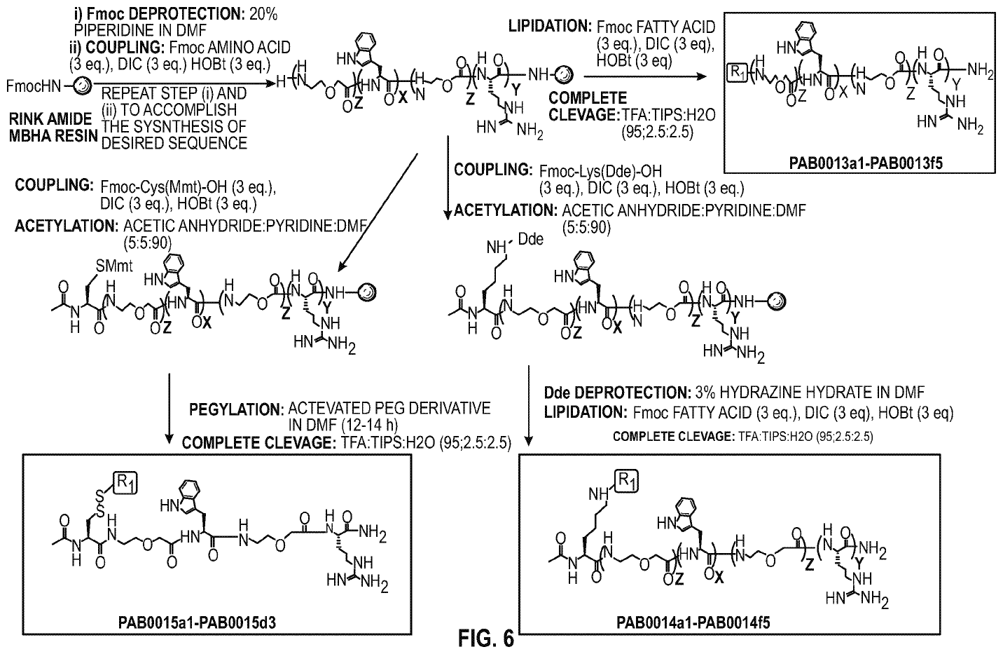

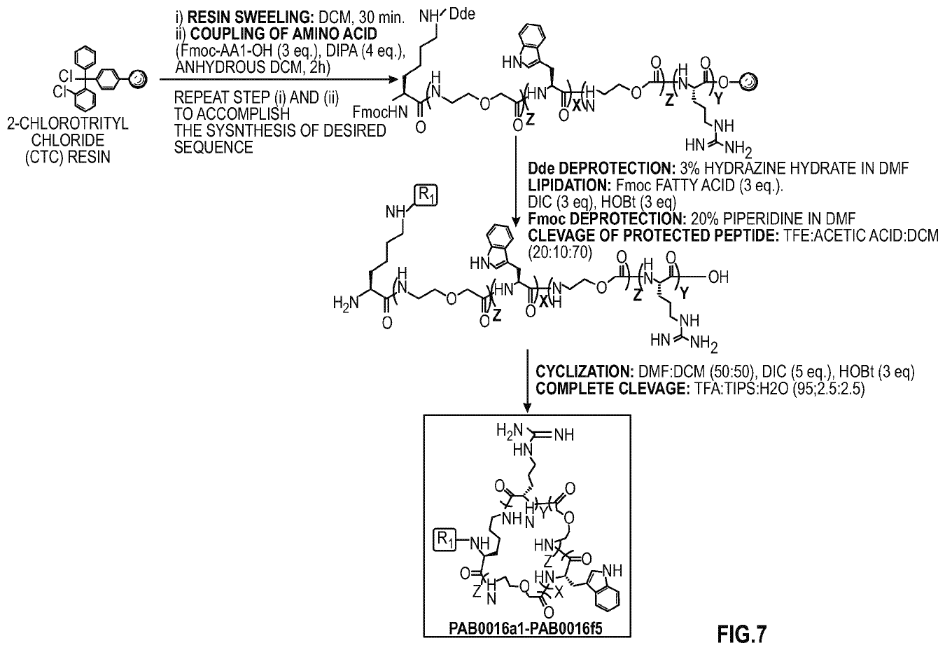

### DETAILED DESCRIPTION

The present invention can be understood more readily by reference to the following detailed description of the invention and the Examples and Figures included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Throughout the description and claims of this specification the word “comprise” and other forms of the word, such as “comprising” and “comprises,” means including but not limited to, and is not intended to exclude, for example, other additives, components, integers, or steps.

As used herein, the terms “optional” or “optionally” means that the subsequently described event or circumstance can or cannot occur, and that the description includes instances where said event or circumstance occurs and instances where it does not.

The term “hybrid peptide” is used to represent a peptide, which consists of a combination of the linear and cyclic peptides. The peptide library was screened for their biophysiochemical properties, such as binding affinity for siRNA, particle size and surface charges of peptide/siRNA complex, and the stability of the complex in the presence of serum, and the siRNA release profile to establish a structure-function relationship.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

### Compositions and Methods

Disclosed are linear, cyclic and/or hybrid peptides containing varying ratios of hydrophobic W and positively charged R residues that transport siRNA inside the cells efficiently and subsequently promote gene silencing efficiency with minimal cytotoxicity.

Disclosed are linear, cyclic and/or hybrid peptides containing the optimum ratio of hydrophobic W and positively charged R residues that transport CRISPR inside the cells efficiently and subsequently promote gene editing with minimal cytotoxicity.

The disclosed compositions can include linear, cyclic, and/or hybrid linear/cyclic peptides containing natural or non-natural positively-charged amino acids, hydrophobic residues, and/or cysteine residues for use as transfection delivery systems. This invention includes fifteen classes of peptides. This invention includes cysteine residues that can form disulfide bonds, modifications, such as substitution of L-amino acid with D-amino acids to avoid proteolytic enzymes, substitution of positively charge arginine or hydrophobic residues with non-natural amino acids, have significantly higher stability, loading efficiency, less toxicity, and effective silencing. Furthermore, the disulfide bridge can be reduced in the presence of glutathione, possibly releasing encapsulated siRNA in the peptide/siRNA complex. The Inventors engineered and characterized peptide carriers containing tryptophan, arginine, and/or cysteine for binding with siRNA, CRISPR, protection against enzymatic degradation, cellular internalization, silencing or gene editing.

The peptides were used in different ratios with siRNA for comparative studies in cancer cells. Furthermore, the effect of hydrophobic modification of the formulation of each peptide/siRNA complex was also evaluated. Inventors characterized the size and surface electrical charge of the complexes formed via ionic interaction between the peptides and siRNA, to confirm complex formation and neutralization of siRNA negative charge, and to evaluate the physical characteristics of the complex. Inventors then analyzed the binding affinity of the peptides to siRNA to determine the effect of peptide and the size of the conjugate on the inter-ionic interaction. The ability of the peptides in protecting siRNA against early enzymatic degradation was investigated by exposing the complexes to fetal bovine serum, and the toxicity of the peptides was explored in different human cancer cell lines. Finally, the efficiency of the peptides in internalizing siRNA into different human cancer cells was studied, which was confirmed by silencing efficiency for specific proteins.

Compounds of the inventive concept represent a new class of transfection delivery agents. The structures of these series of compounds are different than those of current delivery agents. The used amino acids, peptide sequence, examples of their structures, in vitro siRNA binding affinity, cytotoxicity, siRNA delivery, CRISPR delivery, silencing potential, and gene editing potential are summarized herein.

The balance of amphipathic character, density, and position of positive charges and hydrophobic moieties in the conformationally constrained structures of peptides generated an efficient transfecting agent.

The data indicated that several peptides demonstrated potential applications as transfecting agents.

Disclosed herein are compounds, compositions, methods for making and using such compounds and compositions. In specific aspects, disclosed are novel peptide-based systems containing hydrophobic amino acids (e.g., tryptophan), charged amino acids (e.g., arginine), lysine linker, and/or sulfur-containing amino acids (e.g., cysteine), which can be used as nucleic acid transfecting agents or protein delivery in living cells.

A preferred sequence containing hydrophobic amino acids (e.g., tryptophan), charged amino acids (e.g., arginine), connected through various chemically diverse linker such as beta-alanine and/or ethoxy acetic acid (amino-PEG1-acid) and lipidated on N-terminal amine or lysine side chain or PEGylated (polyethylene glycol with different lengths) through cysteine side chain.

Disclosed herein are peptide sequences having the formula:

(R^{z})-(L¹)-(R^{a})ₙ-(L²)-(R^{b})ₙ-(L³)-R^{x},

wherein one of R^{a} or R^{b} represents a hydrophobic amino acid residue, and the other represents a charged amino acid residue,
n is in each case selected from 1-10, preferably 1-8, 1-6, 1-4, 1-2, 2-8, 2-6, 2-4, 3-8, 3-7, 3-6, 3-5 or 3-4,
L¹, L², and L³ are each independently selected from null or a linker,
R^{z} is null, a lipophilic moiety, a PEG moiety, a lysine amino acid residue, or a cysteine amino acid residue,
R^{x} is NH₂, or forms a bond to R^{z};
wherein said lysine residue may further comprise a lipophilic moiety, a PEG moiety, and/or may form a ring with any of R^{a}, R^{L2}, R^{b} or R^{L3},
wherein said cysteine residues may further comprise a lipophilic moiety, a PEG moiety, and/or may form a ring with any of R^{a}, L², R^{b} or L³,

For embodiments in which n is greater than 1, each of the R^{a} and R^{b} residues may be the same, or may be different. In some embodiments, it is preferred than each R^{a} residue is the same, and each R^{b} residue is the same.

Suitable groups for L¹, L², and L³ include groups having the formula:

—[NH—[CH₂]ₐ—X—[CH₂]_{b}—C(═O)]_{c}—,

wherein a is selected from 0-5
b is selected from 0-5, and
c is selected from 1-10, preferably 1-8, 1-6, 1-5, 1-4, 1-3 or 1-2.
X is null or O.

In some embodiments, a is 2, X is null and b is 0, while in other embodiments, a is 2, X is O, and b is 1.

In some embodiments c is 1, and in others c is 2.

In some embodiments it is preferred than L¹, L², and L³ are each present, and are each the same group. In other embodiments, L¹ is null and L² and L³ are the same.

Suitable lipophilic moieties include fatty acids, phospholipids, cholesterol, or other hydrophobic residues. In some embodiments R^{z} has the formula:

CH₃(CH₂)_{d}C(═O)—,

wherein d is from 5-25, preferably 5-20, 5-15, 5-12, 8-25, 8-20, or 8-15. When R^{z} is cholesterol, it may be linked to L¹ or R^{a} via a carbamate bond.

PEG moieties can have an average molecular weight from 100-10,000, from 100-5,000, from 100-2,500, from 100-1,500, from 100-1,000, from 250-5,000, from 500-2,500, from 1,500-3,000, or from 2,000-5,000.

When the PEG is present on a cysteine residue, it may be covalently linked via a disulfide bond. When PEG is present on a lysine residue, it may be covalently linked via an amide or carbonate bond at either the α-nitrogen or the side chain nitrogen.

When R^{z} is a lysine residue, it may form a ring with R^{b} either at the α-nitrogen or the side chain nitrogen.

Disclosed herein are peptide sequences including Class I: linear (Xₙ₋₍β-alanine)ₙ-Yₙ-(β-alanine)ₙ), linear (Yₙ₋₍β-alanine)ₙ-Xₙ-(β-alanine)ₙ), cyclic [Xₙ₋₍β-alanine)ₙ-Yₙ-(β-alanine)ₙ], cyclic [Yₙ₋₍β-alanine)ₙ-Xₙ-(β-alanine)ₙ], lipophilic moiety-Yₙ-(β-alanine)ₙ-Xₙ-(β-alanine)ₙ, linear lipophilic moiety-(β-alanine)ₙ-Yₙ-(β-alanine)ₙ-Xₙ, linear lipophilic moiety-Xₙ-(β-alanine)ₙ-Yₙ-(β-alanine)ₙ, or linear lipophilic moiety-(β-alanine)ₙ-Xₙ-(β-alanine)ₙ-Yₙ; Class II: linear lipophilic moiety-K—Yₙ-(β-alanine)ₙ-Xₙ-(β-alanine)_{z}, linear lipophilic moiety-K-(β-alanine)ₙ-Yₙ-(β-alanine)ₙ-Xₙ, linear lipophilic moiety-K—Xₙ-(β-alanine)ₙ-Yₙ-(β-alanine)_{z}, or linear lipophilic moiety-K-(β-alanine)ₙ-Xₙ-(β-alanine)ₙ-Yₙ; Class III: cyclic lipophilic moiety-[K—Yₙ-(β-alanine)ₙ-Xₙ-(β-alanine)_{z}], cyclic lipophilic moiety-[K-(β-alanine)ₙ-Yₙ-(β-alanine)ₙ-Xₙ], cyclic lipophilic moiety-[K—Xₙ-(β-alanine)ₙ-Yₙ-(β-alanine)_{z}], or cyclic lipophilic moiety-[K-(β-alanine)ₙ-Xₙ-(β-alanine)ₙ-Yₙ]; Class IV: linear PEG-C—Yₙ-(β-alanine)ₙ-Xₙ-(β-alanine)ₙ, linear PEG-C-(β-alanine)ₙ-Yₙ-(β-alanine)ₙ-Xₙ, PEG-C—Xₙ-(β-alanine)ₙ-Yₙ-(β-alanine)ₙ, PEG-C-(β-alanine)ₙ-Xₙ-(β-alanine)ₙ-Yₙ; Class V: linear lipophilic moiety-(Y)ₙ—(X)ₙ, linear lipophilic moiety-(X)ₙ—(Y)ₙ; Class VI: lipophilic moiety-(K)ₙ—(Y)ₙ—(X)ₙ, linear lipophilic moiety-(K)ₙ—(X)ₙ—(Y)ₙ; Class VII: linear PEG-C—Yₙ—Xₙ, PEG-C—Yₙ—Xₙ, linear PEG-K—Yₙ—Xₙ, or PEG-K—Yₙ-Xₙ; Class VIII: linear lipophilic moiety-PEG1-(X)ₙ-PEG1-(Y)ₙ, linear lipophilic moiety-PEG1-(Y)ₙ-PEG1-(X)ₙ; Class IX: lipophilic moiety-K-PEG1-(X)ₙ-PEG1-(Y)ₙ, linear lipophilic moiety-K-PEG1-(Y)ₙ-PEG1-(X)ₙ; Class X: cyclic lipophilic moiety-[K-PEG1-(X)ₙ-PEG1-(Y)ₙ], cyclic lipophilic moiety-[K-PEG1-(Y)ₙ-PEG1-(X)ₙ]; Class XI: linear lipophilic moiety-C-PEG1-(X)ₙ-PEG1-(Y)ₙ], linear lipophilic moiety-[C-PEG1-(Y)ₙ-PEG1-(X)ₙ]; Class XII: linear C(XY)ₙC) or cyclic [(C(XY)ₙC)] (through disulfide bridge or N- to C-terminal or both); Class XIII: linear C(X)ₙ(Y)ₙC)] or cyclic [(C(X)ₙ(Y)ₙC)] (through disulfide bridge or N- to C-terminal or both); Class XIV: linear (Y)ₙCXₙC(Y)ₙ, (X)ₙCYₙC(X)ₙ; Class XIV: hybrid cyclic-linear (Y)ₙ[CXₙC](Y)ₙ or hybrid cyclic-linear (X)ₙ[CYₙC](X)ₙ or hybrid cyclic-linear (Y)ₙ[KXₙK](Y)ₙ or hybrid cyclic-linear (X)ₙ[KYₙK](X)ₙ; Class XV: linear (X)ₙ(KYₙK)(X)ₙ or linear (X)ₙ(Yₙ)(X)ₙ wherein X is a positively-charged amino acid, Y is a hydrophobic residue, wherein n=1-10, lipophilic moiety is fatty acid, phospholipid, cholesterol, or other hydrophobic residues.

Other amino acids can be inserted between positively charged, between hydrophobic residues, or between positively charged and hydrophobic residues while multiple positively-charged residues or multiple hydrophobic amino acids are next to each other, creating a positively-charged component on one side and a hydrophobic component on the other side. Similar or different positively charged or hydrophobic residues may be in the same peptide. In other words, positively charged amino acids can be the same or different. Similarly, hydrophobic amino acids in the same sequence can be the same or different. The peptides can have hybrid structures with cyclic peptides contain positively-charged residues or hydrophobic residues attached to two linear hydrophobic or positively-charged residues.

Amino acids: Examples of suitable positively-charged amino acids (X-residues) in the linear and cyclic peptides are L-arginine, L-lysine, L-histidine, D-histidine, D-arginine, D-lysine. Furthermore, positively-charged amino acids are L- or D-arginine and L- or D-lysine, ornithine, L- or D-histidine residues with shorter or longer side chains (e.g., C3-Arginine (Agp), C4-Arginine (Agb)), diaminopropionic acid (Dap) and diaminobutyric acid (Dab), amino acids containing free side-chain amino or guanidine groups, and modified arginine and lysine residues.

Examples of suitable hydrophobic amino acids (Y-residues) residues in the linear and cyclic peptides are L-tryptophan, D-tryptophan, L-phenylalanine, D-phenylalanine, L-isoleucine, D-isoleucine, p-phenyl-L-phenylalanine (Bip), 3,3-diphenyl-L-alanine (Dip), 3(2-naphthyl)-L-alanine (NaI), 6-amino-2-naphthoic acid, 3-amino-2-naphthoic acid, 1,2,3,4-tetrahydronorharmane-3-carboxylic acid, 1,2,3,4-tetrahydro-3-isoquinolinecarboxylic acid (Tic-OH), 1,2,3,4-tetrahydro-3-isoquinolinecarboxylic acid, modified D- or L-tryptophan residues like N-alkyl or N-aryl tryptophan, substituted D- or L-tryptophan residues (e.g., 5-hydroxy-L-tryptophan, 5-methoxy-L-tryptophan, 6-chloro-L-tryptophan), fatty acids, cholesterol, other N-heteroaromatic and hydrophobic amino acids, and fatty amino acids (e.g., NH₂—(CH₂)ₓ—COOH, where x=1-20, 5-20, 7-20, 10-20, 12-20, 5-18, 5-15, or 5-12).

In some examples, peptides disclosed in each group are inexpensive, easy-to-use, non-toxic, serum-stable, and efficient transfecting agents for all mammalian cells and in vitro nucleic acid delivery.

Some of the disclosed compounds in Class I are composed of linear and cyclic peptides containing positively-charged residues such as arginine and hydrophobic residues such as tryptophan separated from each other by beta-alanine, such as (Wₙ₋₍β-alanine)ₙ-Rₙ-(β-alanine)ₙ where n=1-10. Examples of compounds in this group are [W₄-βAla-R₄-βAla] and (W₄-βAla-R₄-βAla), wherein W represent L-tryptophan and R represents L-arginine.

Some of the disclosed compounds in Class I are composed of positively-charged residues such as arginine and hydrophobic residues such as tryptophan separated from each other by beta-alanine and fatty acyl chain attached on the N-terminal amine (e g., lipophilic moiety-Wₓ-(β-alanine)_{z}-R_{y}-(β-alanine)_{z} or lipophilic moiety-(β-alanine)_{z}-Wₓ-(β-alanine)_{z}-R_{y} (examples, Table 1).

TABLE 1
Linear peptides containing positively-charged residues such as arginine and
hydrophobic residues such as tryptophan separated from each other by beta-
alanine and fatty acyl chain conjugated on N-terminal (Class I).

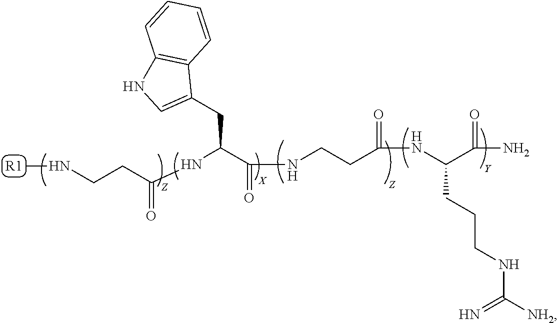

| SEQ | | | | | | |
|---|---|---|---|---|---|---|
| ID | | | | | | |
| NO .: | Sr. No. | Peptide Code | X | Y | Z | R1 |
| 1 | 1 | PAB009a1 | 2 | 2 | 2 | Myristic acid |
| 2 | 2 | PAB009a2 | 2 | 2 | 2 | Palmitic acid |
| 3 | 3 | PAB009a3 | 2 | 2 | 2 | Stearic acid |
| 4 | 4 | PAB009a4 | 2 | 2 | 2 | Oleic acid |
| 5 | 5 | PAB009a5 | 2 | 2 | 2 | Cholesterol |
| 6 | 6 | PAB009b1 | 3 | 3 | 2 | Myristic acid |
| 7 | 7 | PAB009b2 | 3 | 3 | 2 | Palmitic acid |
| 8 | 8 | PAB009b3 | 3 | 3 | 2 | Stearic acid |
| 9 | 9 | PAB009b4 | 3 | 3 | 2 | Oleic acid |
| 10 | 10 | PAB009b5 | 3 | 3 | 2 | Cholesterol |
| 11 | 11 | PAB009c1 | 4 | 4 | 2 | Myristic acid |
| 12 | 12 | PAB009c2 | 4 | 4 | 2 | Palmitic acid |
| 13 | 13 | PAB009c3 | 4 | 4 | 2 | Stearic acid |
| 14 | 14 | PAB009c4 | 4 | 4 | 2 | Oleic acid |
| 15 | 15 | PAB009cS | 4 | 4 | 2 | Cholesterol |
| 116 | 16 | PAB009d1 | 5 | 5 | 2 | Myristic acid |
| 17 | 17 | PAB009d2 | 5 | 5 | 2 | Palmitic acid |
| 18 | 18 | PAB009d3 | 5 | 5 | 2 | Stearic acid |
| 19 | 19 | PAB009d4 | 5 | 5 | 2 | Oleic acid |
| 20 | 20 | PAB009d5 | 5 | 5 | 2 | Cholesterol |
| 21 | 21 | PAB009e1 | 6 | 6 | 2 | Myristic acid |
| 22 | 22 | PAB009e2 | 6 | 6 | 2 | Palmitic acid |
| 23 | 23 | PAB009e3 | 6 | 6 | 2 | Stearic acid |
| 24 | 24 | PAB009e4 | 6 | 6 | 2 | Oleic acid |
| 25 | 25 | PAB009e5 | 6 | 6 | 2 | Cholesterol |
| 26 | 26 | PAB009f1 | 7 | 7 | 2 | Myristic acid |
| 27 | 27 | PAB009f2 | 7 | 7 | 2 | Palmitic acid |
| 28 | 28 | PAB009f3 | 7 | 7 | 2 | Stearic acid |
| 29 | 29 | PAB009f4 | 7 | 7 | 2 | Oleic acid |
| 30 | 30 | PAB009f5 | 7 | 7 | 2 | Cholesterol |

Some of the disclosed compounds in classes II are composed of positively-charged residues such as arginine and hydrophobic residues such as tryptophan separated from each other by beta-alanine and fatty acyl chain attached through lysine side chain (e.g., lipophilic moiety-K—Wₓ-(β-alanine)_{z}-R_{y}-(β-alanine)_{z} or lipophilic moiety-K-(β-alanine)_{z}-Wₓ-(β-alanine)_{z}-R_{y} (Examples, Tables 2). Lipophilic moiety attached to lysine can be at N-terminal or C-terminal.

TABLE 2
Linear peptides containing positively-charged residues such as arginine and hydrophobic
residues such as tryptophan separated from each other by beta-alanine and fatty acyl
chain or cholesterol conjugated on the side chain of the N-terminal lysine residue (Class II).

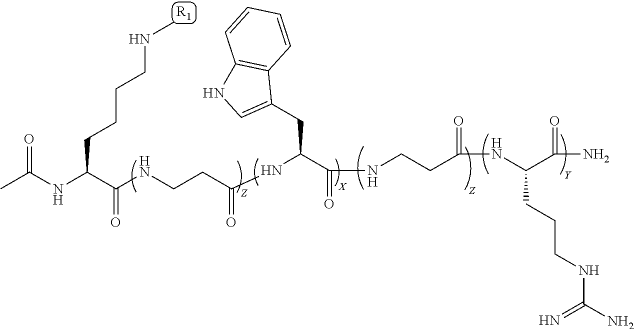

| SEQ | | | | | | |
|---|---|---|---|---|---|---|
| ID | | | | | | |
| NO .: | Sr. No. | Peptide Code | X | Y | Z | R1 |
| 31 | 1 | PAB0010a1 | 2 | 2 | 2 | Myristic acid |
| 32 | 2 | PAB0010a2 | 2 | 2 | 2 | Palmitic acid |
| 33 | 3 | PAB0010a3 | 2 | 2 | 2 | Stearic acid |
| 34 | 4 | PAB0010a4 | 2 | 2 | 2 | Oleic acid |
| 35 | 5 | PAB0010a5 | 2 | 2 | 2 | Cholesterol |
| 36 | 6 | PAB0010b1 | 3 | 3 | 2 | Myristic acid |
| 37 | 7 | PAB0010b2 | 3 | 3 | 2 | Palmitic acid |
| 38 | 8 | PAB0010b3 | 3 | 3 | 2 | Stearic acid |
| 39 | 9 | PAB0010b4 | 3 | 3 | 2 | Oleic acid |
| 40 | 10 | PAB001065 | 3 | 3 | 2 | Cholesterol |
| 41 | 11 | PAB0010c1 | 4 | 4 | 2 | Myristic acid |
| 42 | 12 | PAB0010c2 | 4 | 4 | 2 | Palmitic acid |
| 43 | 13 | PAB0010c3 | 4 | 4 | 2 | Stearic acid |
| 44 | 14 | PAB0010c4 | 4 | 4 | 2 | Oleic acid |
| 45 | 15 | PAB0010c5 | 4 | 4 | 2 | Cholesterol |
| 46 | 16 | PAB0010d1 | 5 | 5 | 2 | Myristic acid |
| 47 | 17 | PAB0010d2 | 5 | 5 | 2 | Palmitic acid |
| 48 | 18 | PAB0010d3 | 5 | 5 | 2 | Stearic acid |
| 49 | 19 | PAB0010d4 | 5 | 5 | 2 | Oleic acid |
| 50 | 20 | PAB0010d5 | 5 | 5 | 2 | Cholesterol |
| 51 | 21 | PAB0010e1 | 6 | 6 | 2 | Myristic acid |
| 52 | 22 | PAB0010e2 | 6 | 6 | 2 | Palmitic acid |
| 53 | 23 | PAB0010e3 | 6 | 6 | 2 | Stearic acid |
| 54 | 24 | PAB0010e4 | 6 | 6 | 2 | Oleic acid |
| 55 | 25 | PAB0010e5 | 6 | 6 | 2 | Cholesterol |
| 56 | 26 | PAB0010f1 | 7 | 7 | 2 | Myristic acid |
| 57 | 27 | PAB0010f2 | 7 | 7 | 2 | Palmitic acid |
| 58 | 28 | PAB0010f3 | 7 | 7 | 2 | Stearic acid |
| 50 | 29 | PAB00104 | 7 | 7 | 2 | Oleic acid |
| 60 | 30 | PAB0010f5 | 7 | 7 | 2 | Cholesterol |

Some of the disclosed compounds in classes III are composed of positively-charged residues such as arginine and hydrophobic residues such as tryptophan separated from each other by beta-alanine cyclized through amide bond formation between lysine side chain and C-terminal carboxylic group and lipidated through N-terminal amine.

TABLE 3
Cyclic lipopeptides containing positively-charged residues such as arginine and
hydrophobic residues such as tryptophan separated from each other by beta-alanine and fatty
acyl chain or cholesteril conjugated through N-terminal amine. (Class III).

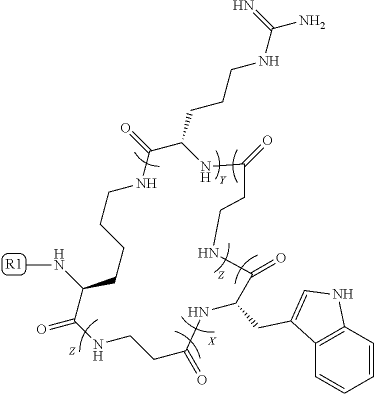

| SEQ | | | | | | |
|---|---|---|---|---|---|---|
| ID NO. | Sr. No. | Peptide Code | X | Y | Z | R1 |
| 61 | 1 | PAB0012a1 | 2 | 2 | 2 | Myristic acid |
| 62 | 2 | PAB0012a2 | 2 | 2 | 2 | Palmitic acid |
| 63 | 3 | PAB0012a3 | 2 | 2 | 2 | Stearic acid |
| 64 | 4 | PAB0012a4 | 2 | 2 | 2 | Oleic acid |
| 65 | 5 | PAB0012a5 | 2 | 2 | 2 | Cholesterol |
| 66 | 6 | PAB0012b1 | 3 | 3 | 2 | Myristic acid |
| 67 | 7 | PAB0012b2 | 3 | 3 | 2 | Palmitic acid |
| 68 | 8 | PAB0012b3 | 3 | 3 | 2 | Stearic acid |
| 69 | 9 | PAB0012b4 | 3 | 3 | 2 | Oleic acid |
| 70 | 10 | PAB0012b5 | 3 | 3 | 2 | Cholesterol |
| 71 | 11 | PAB0012c1 | 4 | 4 | 2 | Myristic acid |
| 72 | 12 | PAB0012c2 | 4 | 4 | 2 | Palmitic acid |
| 73 | 13 | PAB0012c3 | 4 | 4 | 2 | Stearic acid |
| 74 | 14 | PAB0012c4 | 4 | 4 | 2 | Oleic acid |
| 75 | 15 | PAB0012c5 | 4 | 4 | 2 | Cholesterol |
| 76 | 16 | PAB0012d1 | 5 | 5 | 2 | Myristic acid |
| 77 | 17 | PAB0012d2 | 5 | 5 | 2 | Palmitic acid |
| 78 | 18 | PAB0012d3 | 5 | 5 | 2 | Stearic acid |
| 79 | 19 | PAB0012d4 | 5 | 5 | 2 | Oleic acid |
| 80 | 20 | PAB0012d5 | 5 | 5 | 2 | Cholesterol |
| 81 | 21 | PAB0012e1 | 6 | 6 | 2 | Myristic acid |
| 82 | 22 | PAB0012e2 | 6 | 16 | 2 | Palmitic acid |
| 83 | 23 | PAB0012e3 | 6 | 6 | 2 | Stearic acid |
| 84 | 24 | PAB0012e4 | 6 | 6 | 2 | Oleic acid |
| 85 | 25 | PAB0012e5 | 6 | 6 | 2 | Cholesterol |
| 86 | 26 | PAB0012f1 | 7 | 7 | 2 | Myristic acid |
| 87 | 27 | PAB0012f2 | 7 | 7 | 2 | Palmitic acid |
| 88 | 28 | PAB0012f3 | 7 | 7 | 2 | Stearic acid |
| 89 | 29 | PAB0012f4 | 7 | 7 | 2 | Oleic acid |
| 90 | 30 | PAB0012f5 | 7 | 7 | 2 | Cholesterol |

Some of the disclosed compounds (Class IV) are composed of positively-charged residues such as arginine and hydrophobic residues such as tryptophan separated from each other by beta-alanine and PEGylated through sulfur bridge formation on cysteine side chain (e.g., PEG-C—Wₓ-(β-alanine)_{z}-R_{y}-(β-alanine)_{z} or PEG-C-(β-alanine)_{z}-Wₓ-(β-alanine)_{z}-R_{y} (examples, Table 4). PEG moiety attached through a cysteine can be at N-terminal or C-terminal.

TABLE 4
Linear peptides containing positively-charged residues such as arginine and
hydrophobic residues such as tryptophan separated from each other by beta-alanine and
PEGylated through sulfur bridge formation on cysteine side chain (Class IV).

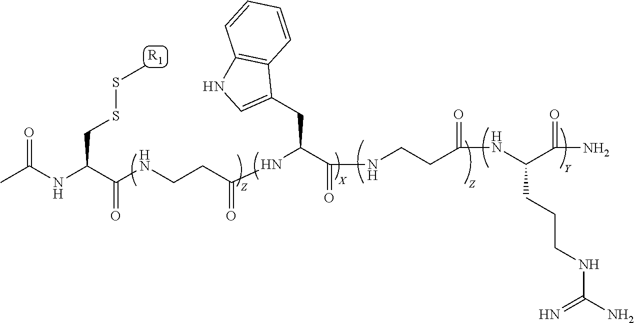

| SEQ | | | | | | |
|---|---|---|---|---|---|---|
| ID. | | | | | | |
| NO. | Sr. No. | Peptide Code | X | Y | Z | R1 |
| 91 | 1 | PAB0011a1 | 2 | 2 | 2 | PEG550 |
| 92 | 2 | PAB0011a2 | 2 | 2 | 2 | PEG2000 |
| 93 | 3 | PAB0011a3 | 2 | 2 | 2 | PEGS000 |
| 94 | 4 | PAB0011a4 | 3 | 3 | 2 | PEG550 |
| 95 | 5 | PAB0011a5 | 3 | 3 | 2 | PEG2000 |
| 96 | 6 | PAB0011b1 | 3 | 3 | 2 | PEG5000 |
| 97 | 7 | PAB0011b2 | 4 | 4 | 2 | PEG550 |
| 98 | 8 | PAB001163 | 4 | 4 | 2 | PEG2000 |
| 99 | 9 | PAB0011b4 | 4 | 4 | 2 | PEG5000 |
| 100 | 10 | PAB001165 | 5 | 5 | 2 | PEG550 |
| 101 | 11 | PAB0011c1 | 5 | 5 | 2 | PEG2000 |
| 1-2 | 12 | PAB0011c2 | 5 | 5 | 2 | PEG5000 |
| 103 | 13 | PAB0011c3 | 6 | 6 | 2 | PEG550 |
| 104 | 14 | PAB0011c4 | 6 | 6 | 2 | PEG2000 |
| 105 | 15 | PAB0011c5 | 6 | 6 | 2 | PEG5000 |
| 106 | 16 | PAB0011d1 | 7 | 7 | 2 | PEG550 |
| 107 | 17 | PAB0011d2 | 7 | 7 | 2 | PEG2000 |
| 108 | 18 | PAB0011d3 | 7 | 7 | 2 | PEG5000 |

Some of the disclosed compounds (Class V) are composed of positively-charged residues such as arginine and hydrophobic residues such as tryptophan with conjugation with fatty acids, cholesterol or phospholipids on N-terminal amine (examples, Table 5). The arginine residues can be at C-terminal or N-terminal.

TABLE 5
Lipopeptides composed of positively-charged residues such
as arginine and hydrophobic residues such
as tryptophan with fatty acylation on N-terminal (Class V).

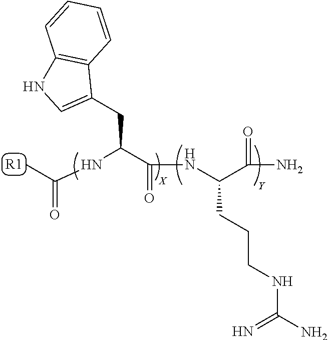

| SEQ | | | | | |
|---|---|---|---|---|---|
| ID. | | | | | |
| NO. | Sr. No. | Peptide Code | X | Y | R1 |
| 109 | 1 | PAB006a1 | 2 | 2 | Myristic acid |
| 110 | 2 | PAB006a2 | 2 | 2 | Palmitic acid |
| 111 | 3 | PAB006a3 | 2 | 2 | Stearic acid |
| 112 | 4 | PAB006a4 | 2 | 2 | Oleic acid |
| 113 | 5 | PAB006a5 | 2 | 2 | Cholesterol |
| 114 | 6 | PAB006b1 | 3 | 3 | Myristic acid |
| 115 | 7 | PAB00662 | 3 | 3 | Palmitic acid |
| 116 | 8 | PAB006b3 | 3 | 3 | Stearic acid |
| 117 | 9 | PAB006b4 | 3 | 3 | Oleic acid |
| 118 | 10 | PAB006b5 | 3 | 3 | Cholesterol |
| 119 | 11 | PAB006c1 | 4 | 4 | Myristic acid |
| 120 | 12 | PAB006c2 | 4 | 4 | Palmitic acid |
| 121 | 13 | PAB006c3 | 4 | 4 | Stearic acid |
| 122 | 14 | PAB006c4 | 4 | 4 | Oleic acid |
| 123 | 15 | PAB006c5 | 4 | 4 | Cholesterol |
| 124 | 16 | PAB006d1 | 5 | 5 | Myristic acid |
| 125 | 17 | PAB006d2 | 5 | 5 | Palmitic acid |
| 126 | 18 | PAB006d3 | 5 | 5 | Stearic acid |
| 127 | 19 | PAB006d4 | 5 | 5 | Oleic acid |
| 128 | 20 | PAB006d5 | 5 | 5 | Cholesterol |
| 129 | 21 | PAB006e1 | 6 | 6 | Myristic acid |
| 130 | 22 | PAB006e2 | 6 | 6 | Palmitic acid |
| 131 | 23 | PAB006e3 | 6 | 6 | Stearic acid |
| 132 | 24 | PAB006e4 | 6 | 6 | Oleic acid |
| 133 | 25 | PAB006e5 | 6 | 6 | Cholesterol |
| 134 | 26 | PAB006f1 | 7 | 7 | Myristic acid |
| 135 | 27 | PAB006f2 | 7 | 7 | Palmitic acid |
| 135 | 28 | PAB006f3 | 7 | 7 | Stearic acid |
| 137 | 29 | PAB006f4 | 7 | 7 | Oleic acid |
| 138 | 30 | PAB006f5 | 7 | 7 | Cholesterol |

Some of the disclosed compounds (Class VI) are composed of positively-charged residues such as arginine and hydrophobic residues such as tryptophan and conjugation with fatty acids, cholesterol, or phospholipids attached through the side chain of the N-terminal lysine residue. (examples, Table 6). The lysine residue can be at C-terminal or N-terminal.

TABLE 6
Lipopeptides composed of positively-charged residues such as
arginine and hydrophobic residues such as tryptophan
with fatty acyl chain attached on the side chain
of the N-terminal lysine residue (Class VI).

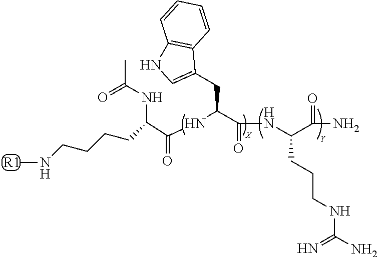

| SEQ | | | | | |
|---|---|---|---|---|---|
| ID. | | | | | |
| NO. | Sr. No. | Peptide Code | X | Y | R1 |
| 139 | 1 | PAB007a1 | 2 | 2 | Myristic acid |
| 140 | 2 | PAB007a2 | 2 | 2 | Palmitic acid |
| 141 | 3 | PAB007a3 | 2 | 2 | Stearic acid |
| 142 | 4 | PAB007a4 | 2 | 2 | Oleic acid |
| 143 | 5 | PAB007a5 | 2 | 2 | Cholesterol |
| 144 | 6 | PAB007b1 | 3 | 3 | Myristic acid |
| 145 | 7 | PAB007b2 | 3 | 3 | Palmitic acid |
| 146 | 8 | PAB00763 | 3 | 3 | Stearic acid |
| 147 | 9 | PAB007b4 | 3 | 3 | Oleic acid |
| 148 | 10 | PAB007b5 | 3 | 3 | Cholesterol |
| 149 | 11 | PAB007c1 | 4 | 4 | Myristic acid |
| 150 | 12 | PAB007c2 | 4 | 4 | Palmitic acid |
| 151 | 13 | PAB007c3 | 4 | 4 | Stearic acid |
| 152 | 14 | PAB007c4 | 4 | 4 | Oleic acid |
| 153 | 15 | PAB007c5 | 4 | 4 | Cholesterol |
| 154 | 16 | PAB007d1 | 5 | 5 | Myristic acid |
| 155 | 17 | PAB007d2 | 5 | 5 | Palmitic acid |
| 156 | 18 | PAB007d3 | 5 | 5 | Stearic acid |
| 157 | 19 | PAB007d4 | 5 | 5 | Oleic acid |
| 158 | 20 | PAB007d5 | 5 | 5 | Cholesterol |
| 159 | 21 | PAB007e1 | 6 | 6 | Myristic acid |
| 160 | 22 | PAB007e2 | 6 | 6 | Palmitic acid |
| 161 | 23 | PAB007e3 | 6 | 6 | Stearic acid |
| 162 | 24 | PAB007e4 | 6 | 6 | Oleic acid |
| 163 | 25 | PAB007e5 | 6 | 6 | Cholesterol |
| 164 | 26 | PAB007f1 | 7 | 7 | Myristic acid |
| 165 | 27 | PAB007f2 | 7 | 7 | Palmitic acid |
| 166 | 28 | PAB007f3 | 7 | 7 | Stearic acid |
| 314 | 29 | PAB007f4 | 7 | 7 | Oleic acid |
| 315 | 30 | PAB007f5 | 7 | 7 | Cholesterol |

Some of the disclosed compounds (Class VII) are composed of positively-charged residues such as arginine and hydrophobic residues such as tryptophan and PEGylated through sulfur bridge formation on the side chain of the cysteine residue (examples, Table 7). The cysteine residue can be at C-terminal or N-terminal.

TABLE 7
Peptides composed of positively-charged residues such as arginine
and hydrophobic residues such as tryptophan, PEGylated
through sulfur bridge formation on the side chain of
the N-terminal cysteine residue (Class VII).

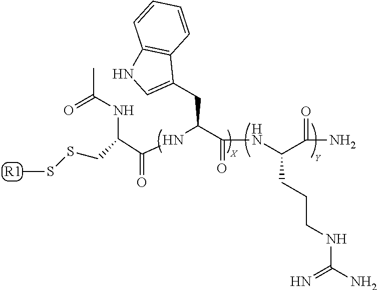

| SEQ | | | | | |
|---|---|---|---|---|---|
| ID. | | | | | |
| NO. | Sr. No. | Peptide Code | X | Y | R1 |
| 167 | 1 | PAB008a1 | 2 | 2 | PEG550 |
| 168 | 2 | PAB008a2 | 2 | 2 | PEG2000 |
| 169 | 3 | PAB008a3 | 2 | 2 | PEG5000 |
| 170 | 4 | PAB008a4 | 3 | 3 | PEG550 |
| 171 | 5 | PAB008aS | 3 | 3 | PEG2000 |
| 172 | 6 | PAB008b1 | 3 | 3 | PEG5000 |
| 173 | 7 | PAB008b2 | 4 | 4 | PEGS50 |
| 174 | 8 | PAB008b3 | 4 | 4 | PEG2000 |
| 175 | 9 | PAB008b4 | 4 | 4 | PEG5000 |
| 176 | 10 | PAB008b5 | 5 | 5 | PEG550 |
| 177 | 11 | PAB008c1 | 5 | 5 | PEG2000 |
| 178 | 12 | PAB008c2 | 5 | 5 | PEG5000 |
| 179 | 13 | PAB008c3 | 6 | 6 | PEG550 |
| 180 | 14 | PAB008c4 | 6 | 6 | PEG2000 |
| 181 | 15 | PAB008c5 | 6 | 6 | PEG5000 |
| 182 | 16 | PAB008d1 | 7 | 7 | PEG550 |
| 183 | 17 | PAB008d2 | 7 | 7 | PEG2000 |
| 184 | 18 | PAB008d3 | 7 | 7 | PEG5000 |

Some of the disclosed compounds (Class VIII) are composed of positively-charged residues such as arginine and hydrophobic residues such as tryptophan with ethoxy acetic acid (amino-PEG1-acid) as linker (examples, Table 8). The lipophilic moiety can be at N-terminal or C-terminal.

TABLE 8
Lipopeptides containing positively-charged residues such as arginine and
hydrophobic residues such as tryptophan with ethoxy acetic acid (amino-PEG1-acid) as
linker and fatty acyl chain or cholesterol attached on the N-terminal amine (Class VIII).

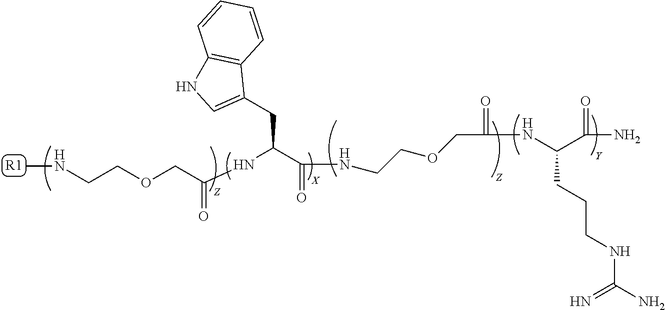

| SEQ. | | | | | | |
|---|---|---|---|---|---|---|
| ID. | | | | | | |
| NO. | Sr. No. | Peptide Code | X | Y | Z | R1 |
| 185 | 1 | PAB0013a1 | 2 | 2 | 2 | Myristic acid |
| 186 | 2 | PAB0013a2 | 2 | 2 | 2 | Palmitic acid |
| 187 | 3 | PAB0013a3 | 2 | 2 | 2 | Stearic acid |
| 188 | 4 | PAB0013a4 | 2 | 2 | 2 | Oleic acid |
| 189 | 5 | PAB0013a5 | 2 | 2 | 2 | Cholesterol |
| 190 | 6 | PAB0013b1 | 3 | 3 | 2 | Myristic acid |
| 191 | 7 | PAB0013b2 | 3 | 3 | 2 | Palmitic acid |
| 192 | 8 | PAB0013b3 | 3 | 3 | 2 | Stearic acid |
| 193 | 9 | PAB0013b4 | 3 | 3 | 2 | Oleic acid |
| 194 | 10 | PAB0013b5 | 3 | 3 | 2 | Cholesterol |
| 195 | 11 | PAB0013c1 | 4 | 4 | 2 | Myristic acid |
| 196 | 12 | PAB0013c2 | 4 | 4 | 2 | Palmitic acid |
| 197 | 13 | PAB0013c3 | 4 | 4 | 2 | Stearic acid |
| 198 | 14 | PAB0013c4 | 4 | 4 | 2 | Oleic acid |
| 199 | 15 | PAB0013c5 | 4 | 4 | 2 | Cholesterol |
| 200 | 16 | PAB0013d1 | 5 | 5 | 2 | Myristic acid |
| 201 | 17 | PAB0013d2 | 5 | 5 | 2 | Palmitic acid |
| 202 | 18 | PAB0013d3 | 5 | 5 | 2 | Stearic acid |
| 203 | 19 | PAB0013d4 | 5 | 5 | 2 | Oleic acid |
| 204 | 20 | PAB0013d5 | 5 | 5 | 2 | Cholesterol |
| 205 | 21 | PAB0013e1 | 6 | 6 | 2 | Myristic acid |
| 206 | 22 | PAB0013e2 | 6 | 6 | 2 | Palmitic acid |
| 207 | 23 | PAB0013e3 | 6 | 6 | 2 | Stearic acid |
| 208 | 24 | PAB0013e4 | 6 | 6 | 2 | Oleic acid |
| 209 | 25 | PAB0013e5 | 6 | 6 | 2 | Cholesterol |
| 210 | 26 | PAB0013f1 | 7 | 7 | 2 | Myristic acid |
| 211 | 27 | PAB0013f2 | 7 | 7 | 2 | Palmitic acid |
| 212 | 28 | PAB0013f3 | 7 | 7 | 2 | Stearic acid |
| 213 | 29 | PAB0013f4 | 7 | 7 | 2 | Oleic acid |
| 214 | 30 | PAB0013f5 | 7 | 7 | 2 | Cholesterol |

Some of the disclosed compounds (Classes IX) are composed positively-charged residues such as arginine and hydrophobic residues such as tryptophan with ethoxy acetic acid (amino-PEG1-acid) as linker and a fatty acyl chain or cholesterol attached through side chain of the lysine residue (examples, Tables 9).

TABLE 9
Lipopeptides containing positively-charged residues such as arginine and
hydrophobic residues such as tryptophan with ethoxy acetic acid (amino-PEG1-acid) as
linker and a fatty acyl chain attached through side chain of the lysine residue (Class IX).

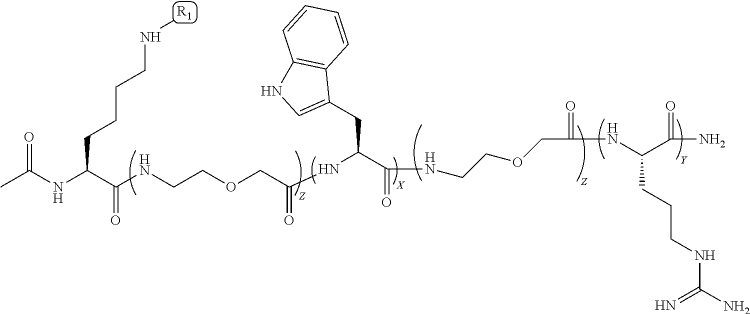

| SEQ | | | | | | |
|---|---|---|---|---|---|---|
| ID. | | | | | | |
| NO. | Sr. No. | Peptide Code | X | Y | Z | R1 |
| 215 | 1 | PAB0014a1 | 2 | 2 | 2 | Myristic acid |
| 216 | 2 | PAB0014a2 | 2 | 2 | 2 | Palmitic acid |
| 217 | 3 | PAB0014a3 | 2 | 2 | 2 | Stearic acid |
| 218 | 4 | PAB0014a4 | 2 | 2 | 2 | Oleic acid |
| 219 | 5 | PAB0014a5 | 2 | 2 | 2 | Cholesterol |
| 220 | 6 | PAB0014b1 | 3 | 3 | 2 | Myristic acid |
| 212 | 7 | PAB0014b2 | 3 | 3 | 2 | Palmitic acid |
| 222 | 8 | PAB0014b3 | 3 | 3 | 2 | Stearic acid |
| 223 | 9 | PAB0014b4 | 3 | 3 | 2 | Oleic acid |
| 224 | 10 | PAB0014b5 | 3 | 3 | 2 | Cholesterol |
| 225 | 11 | PAB0014c1 | 4 | 4 | 2 | Myristic acid |
| 226 | 12 | PAB0014c2 | 4 | 4 | 2 | Palmitic acid |
| 227 | 13 | PAB0014c3 | 4 | 4 | 2 | Stearic acid |
| 228 | 14 | PAB0014c4 | 4 | 4 | 2 | Oleic acid |
| 229 | 15 | PAB0014c5 | 4 | 4 | 2 | Cholesterol |
| 230 | 16 | PAB0014d1 | 5 | 5 | 2 | Myristic acid |
| 231 | 17 | PAB0014d2 | 5 | 5 | 2 | Palmitic acid |
| 232 | 18 | PAB0014d3 | 5 | 5 | 2 | Stearic acid |
| 233 | 19 | PAB0014d4 | 5 | 5 | 2 | Oleic acid |
| 234 | 20 | PAB0014d5 | 5 | 5 | 2 | Cholesterol |
| 235 | 21 | PAB0014e1 | 6 | 6 | 2 | Myristic acid |
| 236 | 22 | PAB0014e2 | 6 | 6 | 2 | Palmitic acid |
| 237 | 23 | PAB0014e3 | 6 | 6 | 2 | Stearic acid |
| 238 | 24 | PAB0014e4 | 6 | 6 | 2 | Oleic acid |
| 239 | 25 | PAB0014e5 | 6 | 6 | 2 | Cholesterol |
| 240 | 26 | PAB0014f1 | 7 | 7 | 2 | Myristic acid |
| 241 | 27 | PAB0014f2 | 7 | 7 | 2 | Palmitic acid |
| 242 | 28 | PAB0014f3 | 7 | 7 | 2 | Stearic acid |
| 243 | 29 | PAB0014f4 | 7 | 7 | 2 | Oleic acid |
| 255 | 30 | PAB0014f5 | 7 | 7 | 2 | Cholesterol |

Some of the disclosed compounds in classes X are composed of positively-charged residues such as arginine and hydrophobic residues such as tryptophan with ethoxy acetic acid (amino-PEG1-acid) as linker and cyclized through amide bond formation between lysine side chain and C-terminal carboxylic group and lipidated through N-terminal amine.

TABLE 10
Cyclic lipopeptides containing positively-charged residues such as
arginine and hydrophobic residues such as tryptophan with ethoxy
acetic acid (amino-PEG1-acid) as linker and fatty acyl
chain conjugated through N-terminal amine (Class X).

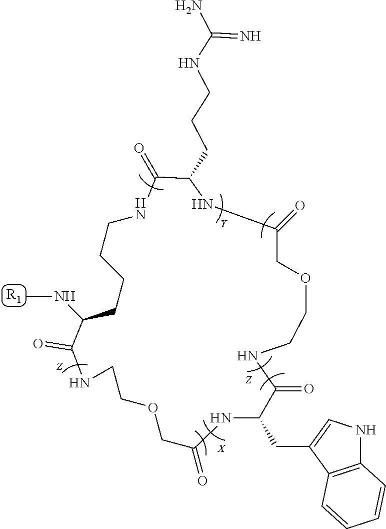

| SEQ | | | | | | |
|---|---|---|---|---|---|---|
| ID. | | | | | | |
| NO. | Sr. No. | Peptide Code | X | V | Z | R1 |
| 256 | 1 | PAB0016a1 | 2 | 2 | 2 | Myristic acid |
| 257 | 2 | PAB0016a2 | 2 | 2 | 2 | Palmitic acid |
| 258 | 3 | PAB0016a3 | 2 | 2 | 2 | Stearic acid |
| 259 | 4 | PAB0016a4 | 2 | 2 | 2 | Oleic acid |
| 260 | 5 | PAB0016a5 | 2 | 2 | 2 | Cholesterol |
| 261 | 6 | PAB0016b1 | 3 | 3 | 2 | Myristic acid |
| 262 | 7 | PAB0016b2 | 3 | 3 | 2 | Palmitic acid |
| 263 | 8 | PAB0016b3 | 3 | 3 | 2 | Stearic acid |
| 264 | 9 | PAB0016b4 | 3 | 3 | 2 | Oleic acid |
| 265 | 10 | PAB0016b5 | 3 | 3 | 2 | Cholesterol |
| 266 | 11 | PAB0016c1 | 4 | 4 | 2 | Myristic acid |
| 267 | 12 | PAB0016c2 | 4 | 4 | 2 | Palmitic acid |
| 268 | 13 | PAB0016c3 | 4 | 4 | 2 | Stearic acid |
| 269 | 14 | PAB0016c4 | 4 | 4 | 2 | Oleic acid |
| 270 | 15 | PAB0016c5 | 4 | 4 | 2 | Cholesterol |
| 271 | 16 | PAB0016d1 | 5 | 5 | 2 | Myristic acid |
| 272 | 17 | PAB0016d2 | 5 | 5 | 2 | Palmitic acid |
| 273 | 18 | PAB0016d3 | 5 | 5 | 2 | Stearic acid |
| 274 | 19 | PAB0016d4 | 5 | 5 | 2 | Oleic acid |
| 275 | 20 | PAB0016d5 | 5 | 5 | 2 | Cholesterol |
| 276 | 21 | PAB0016e1 | 6 | 6 | 2 | Myristic acid |
| 27″ | 22 | PAB0016e2 | 6 | 6 | 2 | Palmitic acid |
| 278 | 23 | PAB0016e3 | 6 | 6 | 2 | Stearic acid |
| 279 | 24 | PAB0016e4 | 6 | 6 | 2 | Oleic acid |
| 280 | 25 | PAB0016e5 | 6 | 6 | 2 | Cholesterol |
| 281 | 26 | PAB0016f1 | 7 | 7 | 2 | Myristic acid |
| 282 | 27 | PAB0016f2 | 7 | 7 | 2 | Palmitic acid |
| 283 | 28 | PAB0016f3 | 7 | 7 | 2 | Stearic acid |
| 284 | 29 | PAB0016f4 | 7 | 7 | 2 | Oleic acid |
| 285 | 30 | PAB0016f5 | 7 | 7 | 2 | Cholesterol |

Some of the disclosed compounds are composed of positively-charged residues such as arginine and hydrophobic residues such as tryptophan with ethoxy acetic acid (amino-PEG1-acid) as linker and PEGylated through cysteine side chain (Table 11).

TABLE 11
Linear peptides containing positively-charged residues such as arginine and
hydrophobic residues such as tryptophan with ethoxy acetic acid (amino-PEG1-acid) as
linker and PEGylated through cysteine side chain (Class XI)

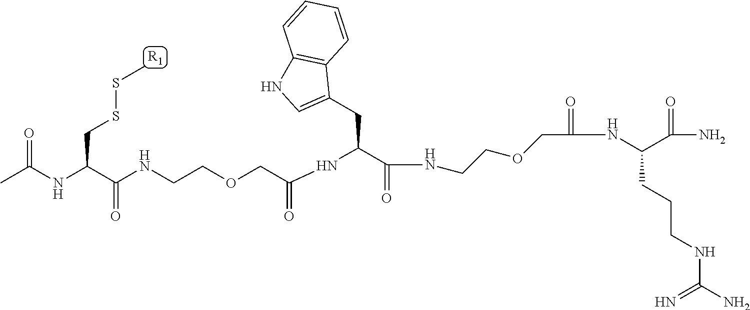

| SEQ | | | | | | |
|---|---|---|---|---|---|---|
| ID. | | | | | | |
| NO. | Sr. No. | Peptide Code | X | Y | Z | R1 |
| 286 | 1 | PAB0015a1 | 2 | 2 | 2 | PEG550 |
| 287 | 2 | PAB0015a2 | 2 | 2 | 2 | PEG2000 |
| 288 | 3 | PAB0015a3 | 2 | 2 | 2 | PEG5000 |
| 290 | 4 | PAB0015a4 | 3 | 3 | 2 | PEG550 |
| 291 | 5 | PAB0015a5 | 3 | 3 | 2 | PEG2000 |
| 292 | 6 | PAB0015b1 | 3 | 3 | 2 | PEG5000 |
| 293 | 7 | PAB0015b2 | 4 | 4 | 2 | PEG550 |
| 294 | 8 | PAB0015b3 | 4 | 4 | 2 | PEG2000 |
| 295 | 9 | PAB0015b4 | 4 | 4 | 2 | PEG5000 |
| 296 | 10 | PAB0015b5 | 5 | 5 | 2 | PEG550 |
| 297 | 11 | PAB0015c1 | 5 | 5 | 2 | PEG2000 |
| 298 | 12 | PAB0015c2 | 5 | 5 | 2 | PEG5000 |
| 299 | 13 | PAB001Sc3 | 6 | 6 | 2 | PEG550 |
| 300 | 14 | PAB0015c4 | 6 | 6 | 2 | PEG2000 |
| 301 | 15 | PAB001Sc5 | 6 | 6 | 2 | PEG5000 |
| 302 | 16 | PAB0015d1 | 7 | 7 | 2 | PEG550 |
| 303 | 17 | PAB001Sd2 | 7 | 7 | 2 | PEG2000 |
| 304 | 18 | PAB0015d3 | 7 | 7 | 2 | PEG5000 |

In one aspect, disclosed are linear, CPs and hybrid cyclic-linear peptides (HCLPs). The concept of using a cyclic peptide-based transfection agent will provide intrinsic property associated with a conformationally constrained structure, such as selectivity to a class of siRNA or cell line, stability, and protection to siRNA from nuclease and serum, and low toxicity. CPs and HCLPs synthesized from natural L-amino acid will be biodegradable, leading to minimum toxicity as compared to lipid or polymer-based transfection agents. The CPs-based transfection agent will be economical as compared to lipid or polymer-based transfecting agents. Structural design of the CPs in the proposed study originated after extensive modification of previously developed classes of CPs containing W and R residues in the cyclic ring ([WR]ₙ; n=4, 5) or R/W residues in the cyclic ring followed by a linear chain of R/W residues (called HCLPs: [R₅K]W₅, and [R₆K]W₆) or R residues in the cyclic ring containing chain of fatty acids ([(R₅(K—C16)₂], [R₅(K—C18)₂]) that demonstrate diverse applications in the non-covalent delivery of siRNA. By controlling and balancing inter-residue electrostatic and hydrophobic interactions with the siRNA molecules, several classes of CPs and HCLPs were synthesized and evaluated with variable cellularization and efficiency in siRNA delivery. Efforts in structural modification provided several lead peptides (cyclic [CR₄W₄C] and hybrid W₄[CR₅C]W₄), which demonstrated high transfection efficiency without toxicity, serum stability as compared to commercial agent lipofectamine Lead peptide [CR₄W₄C] is cyclized through an intramolecular disulfide bridge between cysteine residues, which seems important in the release of siRNA from peptide-siRNA complex in the cytoplasm after being reduced with glutathione. The reduction of the disulfide bridge provides conformational changes in the peptide-siRNA complex, which modulates the release of siRNA. Furthermore, these peptides are internalized independently of endocytic pathways, which is a significant and distinct property as compared to other drug delivery platforms. The unique and intrinsic property of lead peptides to spontaneously translocate across bilayers is distinct from the behavior of well-known, highly cationic CPPs, such as TAT and Arg₉, which do not translocate across synthetic bilayers and instead enter cells primarily by endocytosis. Endosomal entrapment limits nucleic acid transfection efficiency significantly, as the nucleic acids are trapped in endocytic compartments and cannot reach their cytoplasmic or nuclear targets. The other significant advantage of the proposed structures is their high stability and low toxicity. Highly cationic CPPs preferentially interact with particular cell types, have limited serum half-life, show toxicity, and require 9-15 residues of R for efficient cargo delivery. Compared to linear CPPs, which are susceptible to hydrolysis by endogenous peptidases, cyclic peptides are enzymatically more stable and non-toxic. CPs and HCLPs can be used for different nucleic acids (including clustered regularly interspaced short palindromic repeats or CRISPR). Unlike many commercially available transfection agents that could not even be considered for in vivo use, the versatile structure of the proposed peptides offers the possibility of future modifications, e.g., incorporation into gold nanoparticles, conjugation with polyethylene glycol (PEG), and/or targeting moieties for stealth properties and active targeting, respectively, which create clinically relevant delivery systems.

The peptides are amphipathic in nature and conformationally constrained due to the intramolecular disulfide bridge between cysteine residues. Classes XII and XIII comprise of the block (multiple residues) of R and W residues between two cysteines followed by a disulfide bridge, e.g., [CR₄W₄C] (SEQ ID. NO. 305), [C(RW)₄C] (SEQ ID. NO. 306) whereas Class XIV consists of two linear chains of hydrophobic residues on a cyclic peptide of R residues formed through a disulfide bridge, e.g., W₄[CR₅C]W₄ (SEQ ID. NO. 307)) or two linear chains of positively charged residues on a cyclic peptide of W residues formed through a disulfide bridge, e.g., R₄[CW₅C]R₄ (SEQ ID. NO. 308). The R and W residues in Classes XII and XIII could be alternate or all the R residues on one side and W residues on the opposite side, respectively. The cyclic peptides also include N to C cyclic peptides containing R and W residues with R residues on one side and W residues on the opposite side, e.g. [R₄W₄] (SEQ ID. NO. 309) The lead peptides were synthesized using Fmoc/tBu solid-phase chemistry and fully characterized. Complexes were formed using scrambled siRNA and both peptides at a wide range of N/P ratios. The complexes formed with both peptides had a size of approximately 200 nm or lower and showed slightly positive zeta-potential, which increased at higher N/P ratios.

The data indicated that the lead cyclic [CR₄W₄C] (SEQ ID. NO. 305) and hybrid W₄[CR₅C]W₄) (SEQ ID. NO. 307) peptides containing disulfide bridge demonstrated potential applications as transfecting agents. Classes XII and XIII peptides explore both the role of the disulfide bridge and constrained cyclic structure in developing optimal transfecting agents. Class XIV represents HCLP analogs, composed of hydrophobic linear chains attached to a cyclic peptide containing positively charged R residues or linear chains of positively charged residues on a cyclic peptide of W residues.

Some of the disclosed compounds (Class XV) are composed of linear peptides containing positively-charged residues such as arginine and hydrophobic residues such as tryptophan attached directly or through a lysine residue.

The structural modifications in the lead peptides were used to optimize them as efficient vectors for cellular delivery of siRNA through enhancing their interactions with negatively charged siRNA and providing them protection from the nucleases, and mediating the efficient cellular uptake.

Compounds disclosed herein represent a new class of transfection and CRISPR delivery agents. The structures of these series of compounds are different from those of current delivery agents.

Also disclosed is peptide-based universal, robust, and non-toxic transfection agents (TAs) that can deliver siRNA, CRISPR, or proteins efficiently to a wide variety of mammalian cell lines.

Also disclosed are cysteine-containing cyclic amphipathic peptide [CW₅R₅C] (SEQ ID. NO. 305) enclosed with a disulfide bond and hybrid peptide W₄[CR₅C]W₄ (SEQ ID. NO. 307) (composed of positively charged R-containing ring and hydrophobic W-containing side chains), which exhibited significant transfection efficiency (˜80%) while targeting STAT3 mRNA in breast cancer cell line MDA-MB-231.

Additional advantages will be set forth in part in the description that follows, and in part will be obvious from the description or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The peptides can be used as pegylated peptides with or without targeting moieties for nucleic acid or protein delivery.

The peptides can be used as lipidated peptides with or without targeting moieties for nucleic acid or protein delivery.

The peptides can be used as lapidated and pegylated peptides with or without targeting moieties for nucleic acid or protein delivery.

Some of the disclosed peptides contain a disulfide bridge for cyclization and two chains of hydrophobic tryptophan residues. The rationale of this design is based on the data that a cyclic peptide [WR]₅ containing alternating tryptophan (W) and arginine (R) residues (e.g. WRWR) significantly enhanced the cellular uptake of negatively charged phosphopeptides and siRNA (Shirazi A N, Paquin K L, Howlett N G, Mandal D, Parang K. Cyclic peptide-capped gold nanoparticles for enhanced siRNA delivery. Molecules. 2014; 19(9):13319-31. doi: 10.3390/molecules190913319. PubMed PMID: 25170952; PMCID: PMC6271229). Some examples of peptides are [CRWRWRWRWC] (SEQ ID NO: 310), [CRWRWRWRWRWC] (SEQ ID NO: 311), [CRRRRWWWWC] (SEQ ID NO: 312), and [CRRRRRWWWWWC] (SEQ ID NO: 313).

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric.

All amino acid building blocks and preloaded amino acids on the resin used in this study were purchased from AAPPTEC. Other reagents, chemicals, and solvents were procured from Sigma-Aldrich. The final compounds used in further studies were purified with reversed-phase High-performance liquid chromatography from Shimadzu (LC-20AP) using a binary gradient system of acetonitrile 0.1% TFA and water 0.1% TFA and a reversed-phase preparative column (X Bridge BEH130 Prep C18, 10 μm 18×250 μm Waters, Inc). The chemical structure of linear, cyclic, and hybrid cyclic-linear peptides, intermediates, and final products were characterized by high-resolution MALDI-TOF (GT-0264) from Bruker Inc.

The peptides were synthesized using Fmoc/tBu solid-phase synthesis. In brief, the linear peptides were assembled on trityl resin. The amino acids in the sequence were conjugated using Fmoc-amino acid building blocks in the presence of 2-(1H-benzotriazole-1-yl)-1,1,3,3-tetramethyluronium hexafluorophosphate (HBTU), and diisopropylethylamine (DIPEA) in dimethylformamide (DMF). After each coupling, the Fmoc deprotection was performed using 20% (v/v) piperidine in DMF. The progress of the reaction was monitored by matrix-assisted laser desorption/ionization (MALDI) time-of-flight (TOF) analyzer. The resultant peptides were cleaved from the resin, followed by purification and lyophilization. The linear peptides containing cysteine residues were oxidized in the presence of 10% DMSO/Water to introduce disulfide bridges.

Linear, cyclic, hybrid cyclic-linear peptides were synthesized that have hydrophobic positively-charged, and/or cysteine residues as the amino acid sequence in its basic structure. Inventors varied the amino acid constituents in the structure to determine the effectiveness of derived compounds in siRNA binding and delivery and to establish a structure-activity relationship. The strategy was to vary net hydrophobicity and the positive charges of derived compounds based on structure-activity relationships and evaluate siRNA binding, delivery, and silencing potentials. The rationale of current studies was that cancer cells have a higher percentage of glutathione relative to normal cells. Accordingly, disulfide bonds can be reduced in cancer cells to a significantly higher degree than normal cells, providing selective delivery of siRNA to cancer cells.

General method for the synthesis of linear peptides. The synthesis of linear peptides was performed by Fmoc/tBu solid-phase peptide synthesis method. All the peptides were synthesized using Fmoc solid-phase peptide synthesis using appropriate resin and Fmoc-protected amino acids. Fmoc protected amino acids were used as the building blocks for conducting the synthesis of peptides on 0.3 mmol scale. 2-(1H-benzotriazol-1-yl)-1,1,3,3-tetramethyluronium hexafluorophosphate (HBTU)/N,N-diisopropylethylamine (DIPEA) were used as coupling and activating reagents, respectively. Piperidine in N,N-dimethylformamide (DMF) (20%, v/v) was used for Fmoc deprotection. The resultant peptides were cleaved from the resin, and all protecting groups were removed using a cleavage cocktail of TFA/anisole/EDT/thioanisole (90:5:3:2, v/v/v/v) for 3 h. Crude products were precipitated by the addition of cold diethyl ether purified by reverse phase HPLC using a gradient of 0-90% acetonitrile (0.1% TFA) and water (0.1% TFA) over 60 min with C-18 column. Purified peptides were lyophilized to yield a white powder. The chemical structures of all synthesized peptides were elucidated using matrix-assisted laser desorption/ionization (MALDI) time-of-flight (TOF) mass spectroscopy with α-cyano hydroxycinnamic acid (CHCA) as a matrix.

Synthesis of cyclic peptides through N- to C-cyclization. Amino acid loaded on trityl resin and Fmoc-amino acid building blocks were used for the synthesis on a scale of 0.3 mmol. HBTU/DIPEA was used as coupling and activating reagents, respectively. Piperidine in DMF (20% v/v) was used for Fmoc deprotection. The side-chain-protected peptides were detached from the resin by TFE/acetic acid/DCM [2:1:7 (v/v/v)] then subjected to cyclization using 1-hydroxy-7-azabenzotriazole (HOAT) and N,N′-diisopropylcarbodiimide (DIC) in an anhydrous DMF/DCM mixture overnight. All protecting group were removed with cleavage cocktail of TFA/anisole/thioanisole (90:2:5 v/v/v) for 3 h. The crude products were precipitated by the addition of cold diethyl ether purified using reverse-phase HPLC using a gradient of 0-90% acetonitrile (0.1% TFA) and water (0.1% TFA) over 60 min with C-18 column. The purified peptide was lyophilized to yield a white powder (100 mg). The chemical structure of all synthesized peptides was elucidated using matrix-assisted laser desorption/ionization (MALDI) time-of-flight (TOF) mass spectrometer.

Synthesis of cyclic peptides cyclized through a disulfide bridge. About 30 mg of the linear peptide was dissolved in a 10% DMSO-H₂O solution (150 ml). The reaction mixture was stirred for 24-72 h at room temperature in an open round-bottomed flask. The reaction mixture was injected directly in reverse phase HPLC using a gradient of 0-90% acetonitrile (0.1% TFA) and water (0.1% TFA) over 60 min with C-18 column. The purified peptide was lyophilized to yield a white powder (20 mg). The chemical structure of all synthesized peptides was elucidated using matrix-assisted laser desorption/ionization (MALDI) time-of-flight (TOF) mass spectrometer.

The synthesis of linear and cyclic peptides was conducted according to the previously reported procedure by us (Mohammed et al., Comparative molecular transporter properties of cyclic peptides containing tryptophan and arginine residues formed through disulfide cyclization. Molecules 2020, 25(11):E2581. doi: 10.3390/molecules25112581). Parentheses ( ) and brackets represent [ ] linear and cyclic peptides, respectively.

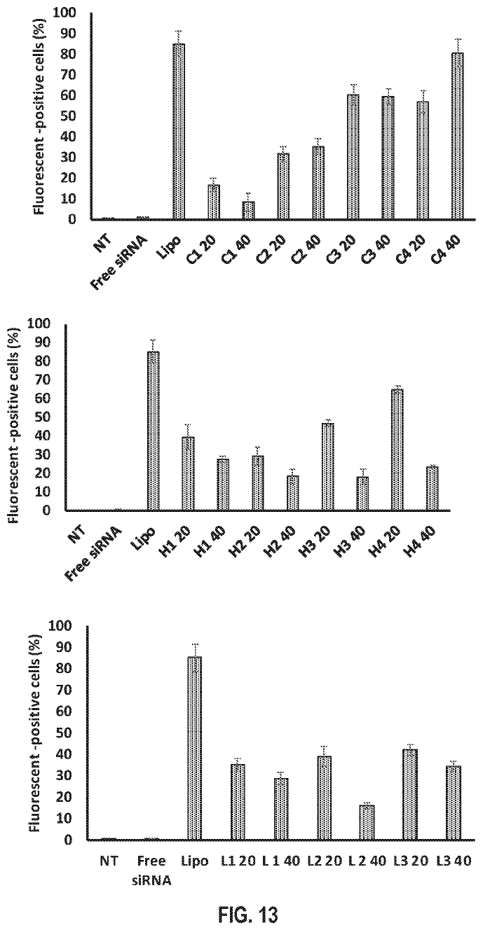

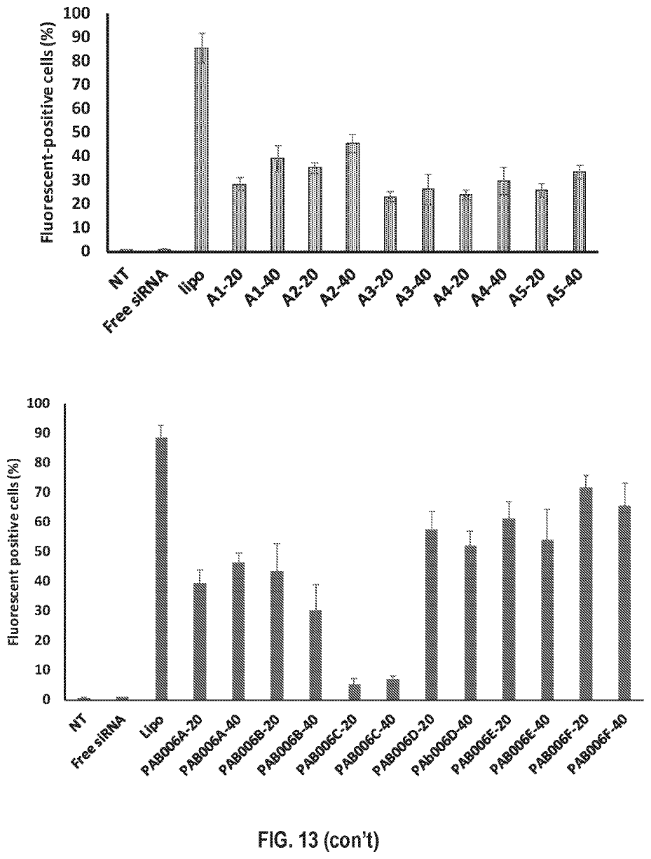

Synthesis of the pegylated cell-penetrating peptide (PEG-CPP). The solid-phase synthesis of the linear peptide sequence was conducted using the standard protocol mentioned before. The desired peptide sequence was assembled on rink amide resin. Orthogonal protected Cys(StBu) residues were incorporated to achieve on-resin sulfur bridge formation. N-Terminal of parent peptide was further extended by coupling 2-Fmoc aminoethoxy acetic acid as a spacer between the parent peptide and another cysteine residue Cys(Mmt), which was installed to pegylate the peptide by exploiting free side chain thiol functionality. The tert-butyl thiol groups were deprotected on resin using 3.8 mmol dithiothreitol (DTT) in 2.5 mL DMF with 0.25 mL DIEA. The reaction proceeded at 60° C. for 20 minutes. The resin was washed thoroughly with DMF and DCM. On-resin disulfide formation reaction was conducted using N-chlorosuccinimide (NCS, 2 eq.) in DMF. Methoxytrityl group was deprotected selectively by repeatedly treating the peptidyl resin with 2% TFA in DCM. The resulted free thiol functionality was used for pegylation of the peptide by reacting with preactivated PEG (mPEG-OPSS; Orthopyridyl disulfide). For on-resin pegylation reaction, 2 eq. of preactivated PEG-SH was added to the peptidyl resin in DMF. The reaction mixture was kept for shaking at room temperature for around 14 hours (FIG. 13). The pegylated peptide was cleaved from the resin and purified by RP-HPLC, and mass was confirmed by Q-TOF-LC-MS.

Complex formation with modified peptides and siRNA. The peptide/siRNA complexes were formed in different N/P ratios for the variety of the in vitro studies conducted according to the previously reported procedure (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel). 2019; 11(4). doi: 10.3390/polym11040703. PubMed PMID: 30999603; PMCID: PMC6523661).

All siRNAs used in this project contain 21-25 base pairs in each strand. The scrambled siRNA used in this set of experiments contains 23 base pairs.

Size and Surface Charges. The efficiency of siRNA carriers largely depends on the size and overall surface charge of the complex. Dynamic light scattering (DLS) was used to determine the size and ζ-potential at different N/P ratios to determine the optimal N/P ratio for a compact complex (50-200 nm) and slightly positive charge.

siRNA binding affinity. To evaluate binding affinity to siRNA, a study was designed based on the quantification of free siRNA using SYBR Green II. Peptides were mixed with siRNA with a wide range of N/P ratios (0.05-40), and the amount of unbound siRNA was quantified by the fluorescent signal of SYBR Green II. The required N/P ratio for 50% binding (BC50) was calculated for each peptide.

Serum Stability. In order to determine the capability of the complexes to protect the siRNA against enzymatic degradation, Inventors exposed different study groups to a diluted fetal bovine serum (FBS) solution, with “naked” siRNA as a positive control, and siRNA exposed to saline as a negative control according to the previously reported procedure (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel). 2019; 11(4) doi: 10.3390/polym11040703. PubMed PMID: 30999603; PMCID: PMC6523661).

The binding affinity for [CR₄W₄C] (SEQ ID. NO. 305) and W₄[CR₅C]W₄ (SEQ ID. NO. 307) showed that approximately 4 and 10 N/P ratios are required for 50% binding. While “naked” siRNA was completely degraded in the presence of serum, both peptides were able to protect siRNA completely after 24 hours of exposure to the serum (as compared to siRNA exposed to saline as negative control) at an N/P ratio of 40. The toxicity of the peptides was evaluated in triple-negative breast cancer (TNBC) cells MDA-MB-231 (as a model cancer cell line) with peptide alone and siRNA complexes, and CCK assay to quantify cell viability after 48 hours.

Protection of siRNA against Enzymatic Degradation. In order to study the capability of the fatty acid-conjugated linear and cyclic peptides to enhance the stability of siRNA in biological environments, samples of unprotected scrambled siRNA (positive control) and peptide/siRNA complexes at a different peptide:siRNA ratios were exposed to fetal bovine serum (FBS) solutions. After completion of complex formation for each formulation, each sample was added to a 25% v/v FBS solution in HBSS, and the mixture was incubated at 37° C. for 24 h. A sample of unprotected siRNA in HBSS was used as a negative control, representing 100% intact siRNA. After the incubation, complexes were dissociated using a 2:3 mixture of heparin (5% solution in normal saline) and EDTA (0.5 mM), and the samples were analyzed using a 1% agarose gel (with 1 μg/mL ethidium bromide) at 70 V for 20 minutes. UV illumination (Gel-Doc system, Bio-Rad; Hercules, CA) was used to visualize the gel, and the intensity of the bands (representing remaining intact siRNA) was quantified by Image J software (FIG. 8).

Cytotoxicity Assays. The cytotoxicity of the peptides was evaluated in a panel of cancer and non-cancerous cell lines. Normal human breast (Hs 578Bst, ATCC™ HTR-125™), kidney (LLCP-K1, ATCC CRL-1392), prostate (RWPE-1; ATCC™ CRL-11609™), colon (CCD-33Co; ATCC™ CRL-1539™), heart (H9C2, ATCC CRL 1446) or endometrial (KC02-44D hTERT, ATCC™ SC-6000™) cell lines were exposed to a wide range of peptide/siRNA concentrations. Due to the popularity of siRNA silencing in cancer research, the toxicity of the same study groups were also evaluated in TNBC cells MDA-MB-231 (ATCC™ HTB-26™), MDA-MB-468 (ATCC™ HTB-132), ovarian SK-OV-3 (ATCC™ HTB-77™), colorectal ht-29 (ATCC™ HTB-38™), and prostate LNCaP (ATCC™ CRL-1740™) cancer cell lines. Cell viability was evaluated by CCK assay after 48 hours of exposure. Neither of the peptides showed significant toxicity in the range of concentration or N/P ratio evaluated.

The human breast cancer cell line MDA-MB-231 was purchased from American Type Culture Collection (ATCC; Manassas, VA). MDA-MB-231 was cultured in the DMEM medium. The media was supplemented with 10% (v/v) fetal bovine serum, 100 U/mL penicillin, and 100 μg/ml streptomycin. Cells were maintained in the normal condition of 37° C. and 5% CO₂ under a humidified atmosphere and were sub-cultured when 80-100% confluent.

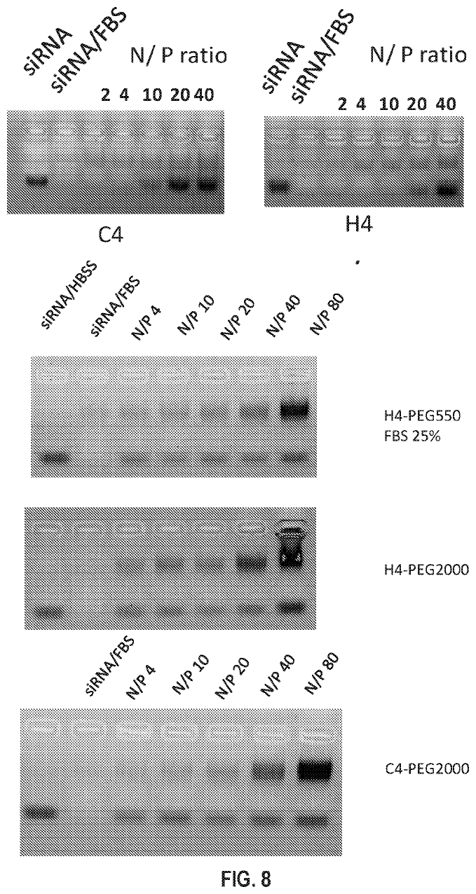

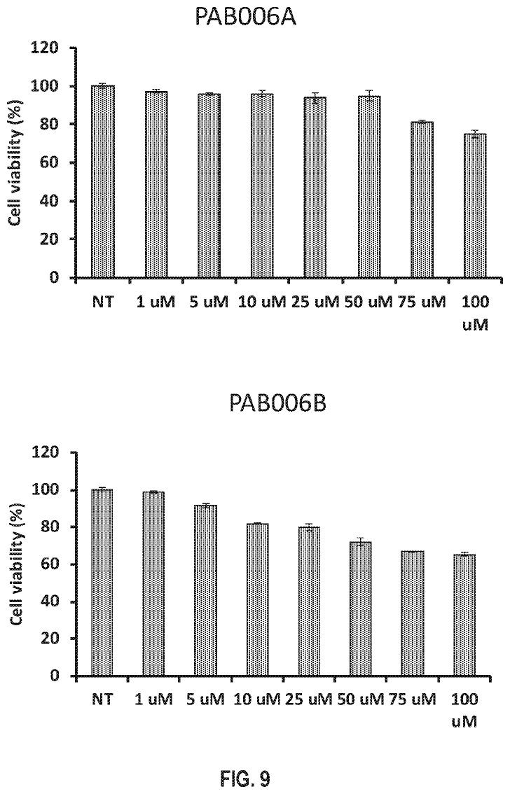

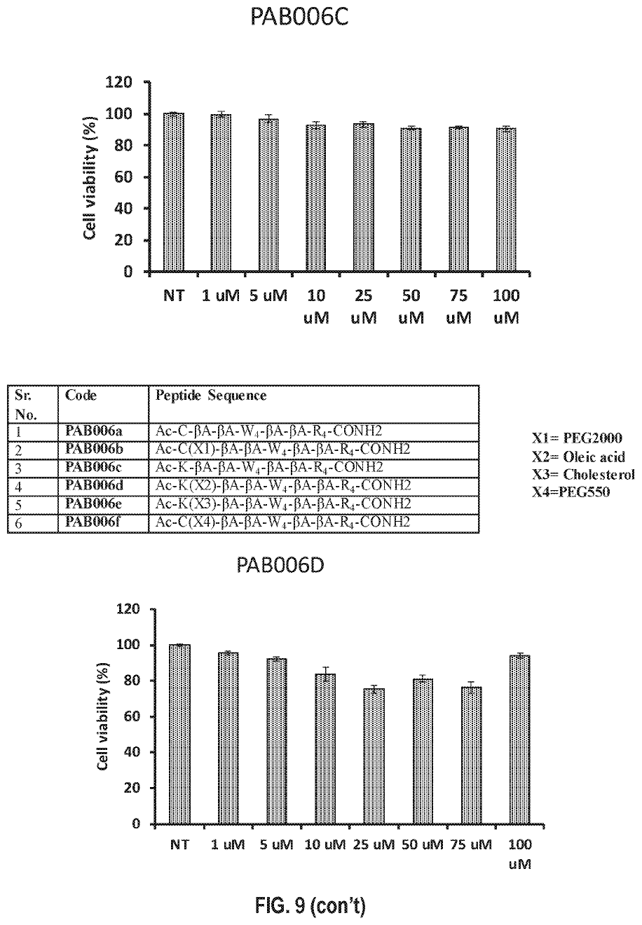

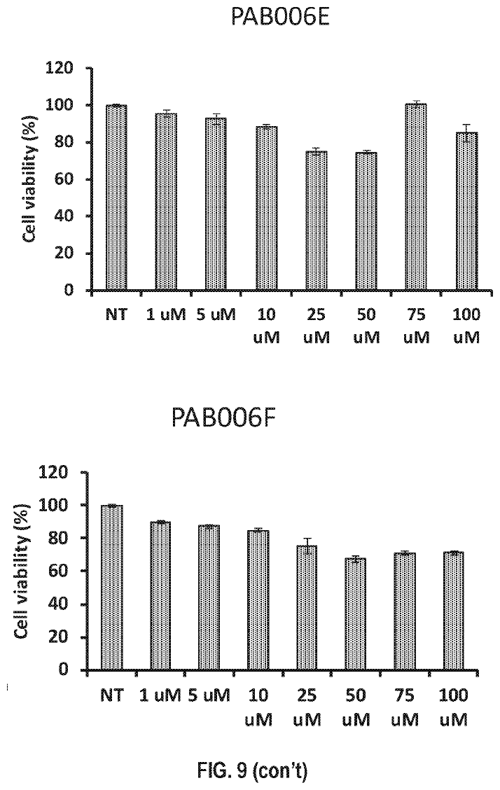

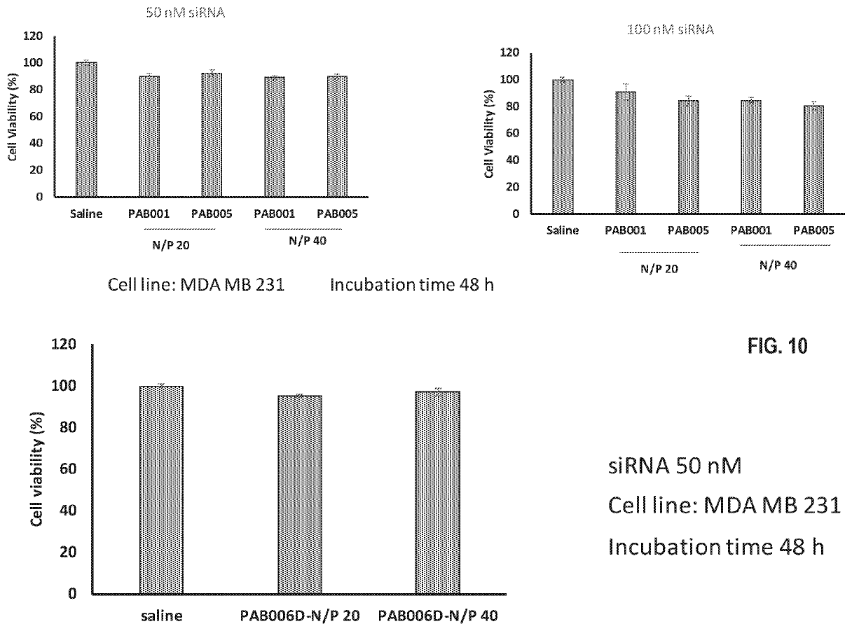

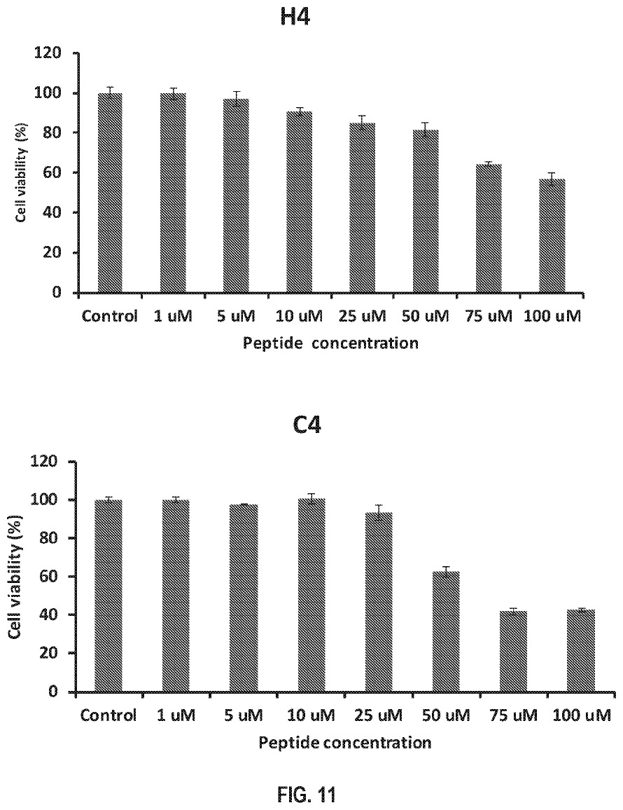

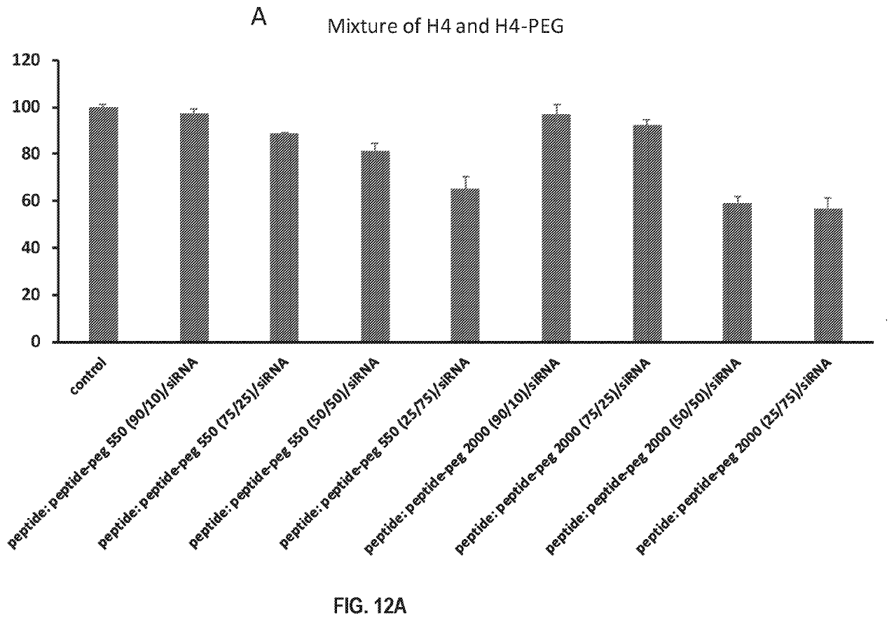

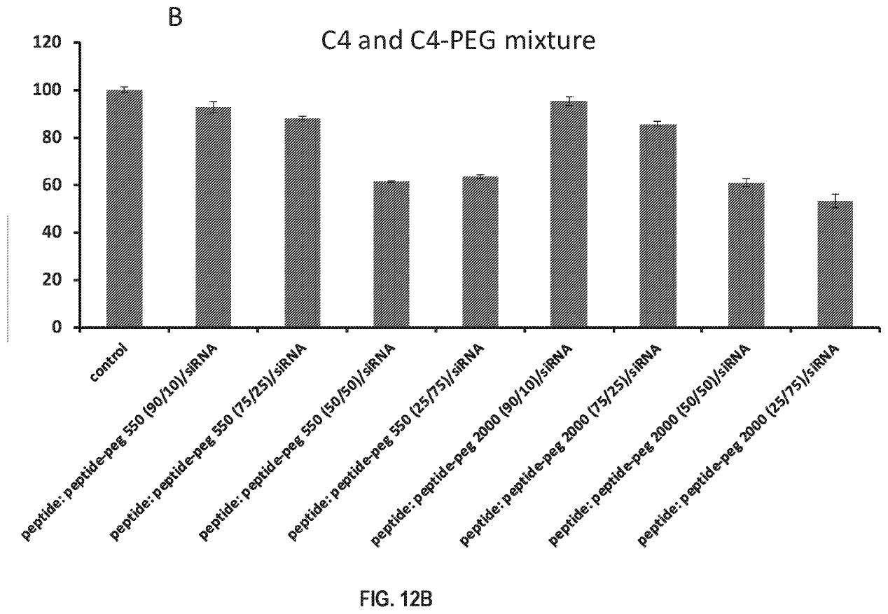

Toxicity of the Peptide/siRNA Complexes: The effect of peptides on the viability of human cell lines was evaluated by exposing the MDA-MB-231 cell lines (FIGS. 9-12) to different concentrations of peptide solution and exposing the cells to peptide/siRNA complexes prepared with different peptides at a various peptide:siRNA ratios according to the previously reported procedure by us (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel). 2019; 11(4). doi: 10.3390/polym11040703. PubMed PMID: 30999603; PMCID: PMC6523661). Confluent cultures (≈800,000 cells/mL) of MDA-MB-231 cancer cell lines were seeded in 96-well plates. After 24 h, cells were exposed to different concentrations of peptide solutions or peptide/siRNA complexes. The final concentration of scrambled siRNA was 36 nM in all cytotoxicity studies performed with peptide/siRNA complexes. The cells were then incubated for 48 hours at 37° C. with controlled CO₂ and humidity. A standard 3-(4,5-dimethylthiazol-2-yl)-2,5-diphenyltetrazolium bromide (MTT) assay was performed after the incubation period to determine the cell viability as a percentage of untreated (NT) cells.

Cellular Uptake. Cellular uptake was quantified using flow cytometry and was evaluated in all the cell lines included in the cytotoxicity study. However, since this method did not confirm cytoplasmic accumulation, confocal microscopy was performed to confirm and visualize the cytoplasmic delivery. Free siRNA and Lipofectamine™ were used as negative and positive controls, respectively (FIG. 13).

The ability of peptides to deliver siRNA into the MDA-MB-231 cells was evaluated using FAM-labeled scrambled siRNA and analysis by flow cytometry (FIG. 13) according to the previously reported procedure by us (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel). 2019; 11(4). doi: 10.3390/polym11040703. PubMed PMID: 30999603; PMCID: PMC6523661). MDA-MB-231 cells were used for these studies and were seeded in 24-well plates (≈200,000 cells per well). After the addition of the siRNA complexes to cell culture media, cells were incubated at 37° C. and standard growth conditions for 24 hours. After the incubation period, and for flow cytometry studies, cells were washed with clear HBSS (×2), trypsinized, and fixed using 3.7% formaldehyde solution. Suspended cells were analyzed with a BD-FACSVerse (BD Biosciences; San Jose, CA) using the FITC channel to quantify cell-associated fluorescence. After each flow cytometry analysis, the percentage of cells with fluorescence signal and the mean fluorescence of the cell population were calculated based on the calibration of the signal gated with non-treated cells (as the negative control), so that the auto-fluorescence would be ≈1% of the population.

The peptides showed significant siRNA uptake, which was significantly higher at a peptide:siRNA ratio of 20:1 and 40:1 after 24 h incubation. The mean fluorescence results and positive fluorescence cells confirmed a similar pattern. The addition of PEGylated peptide (25%) in the native peptide solution enhances the uptake of siRNA.

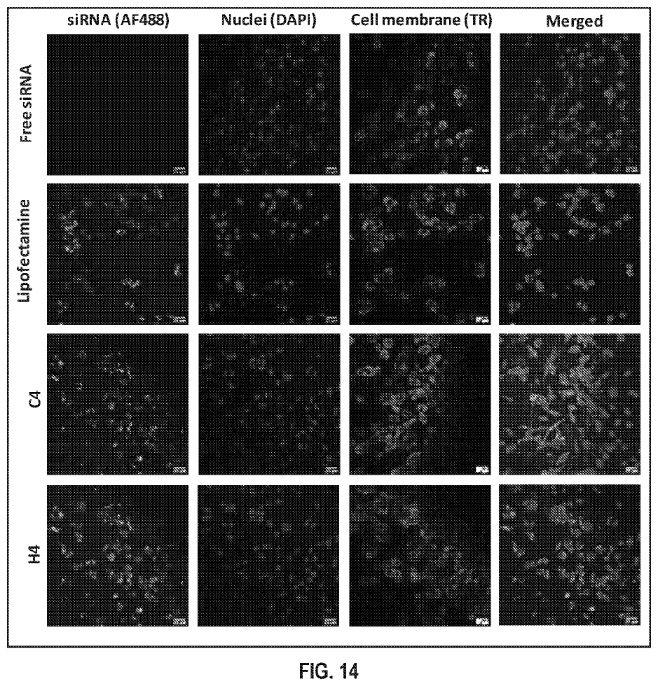

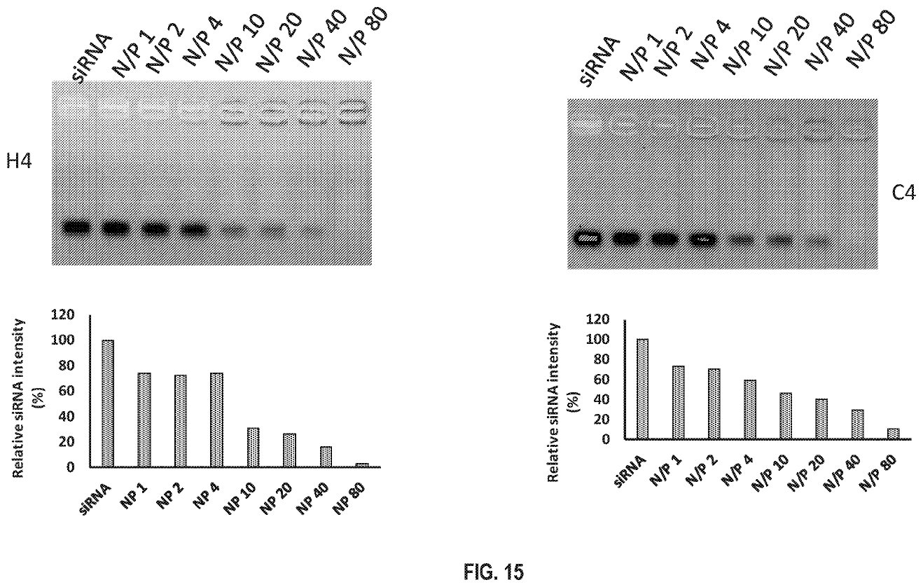

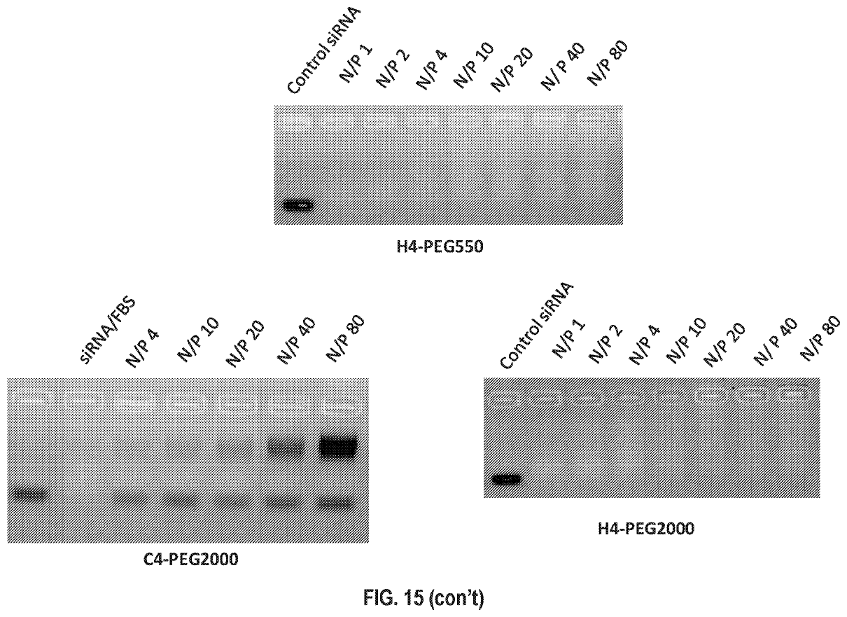

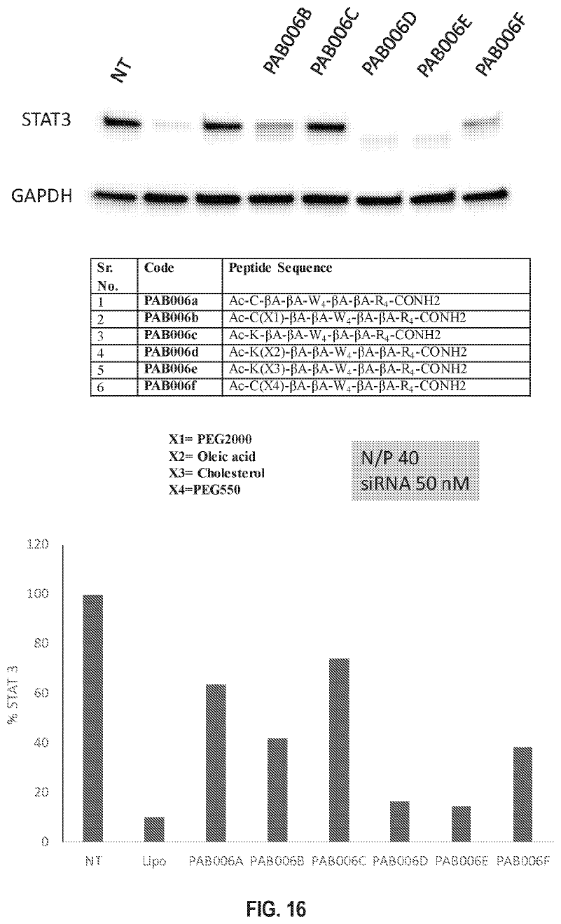

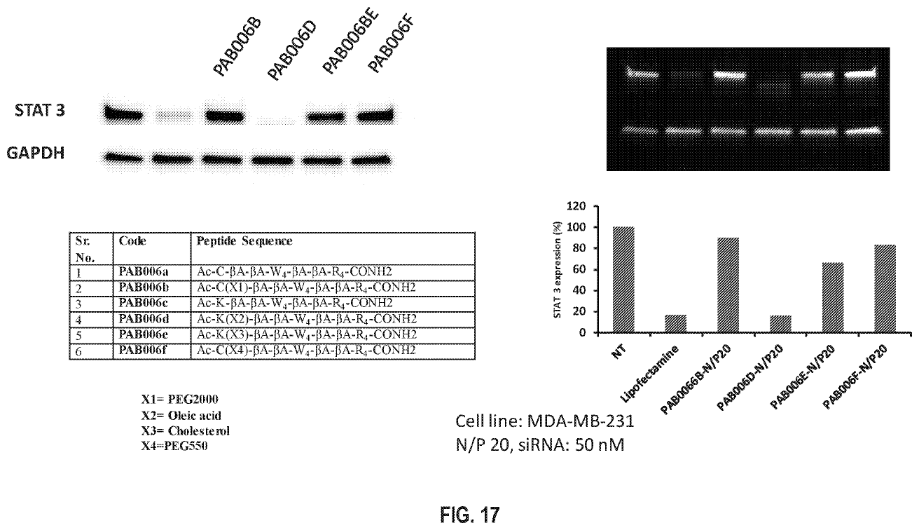

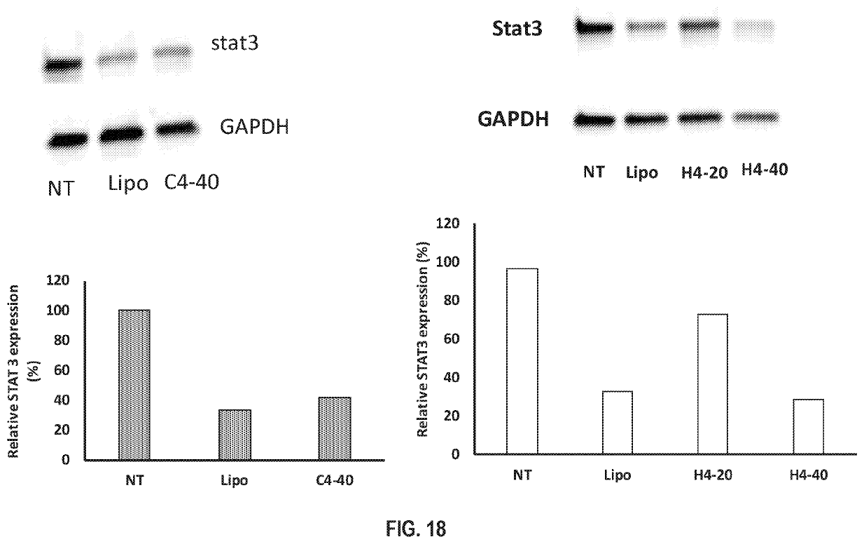

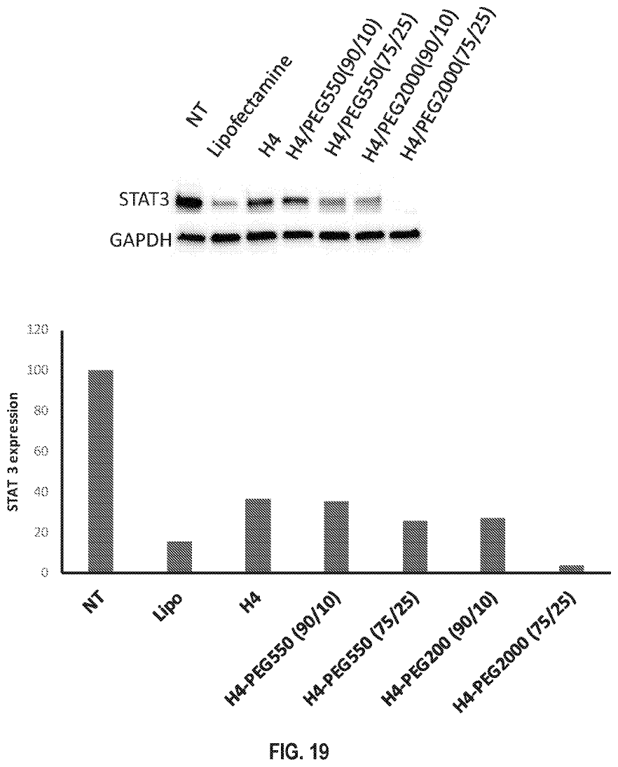

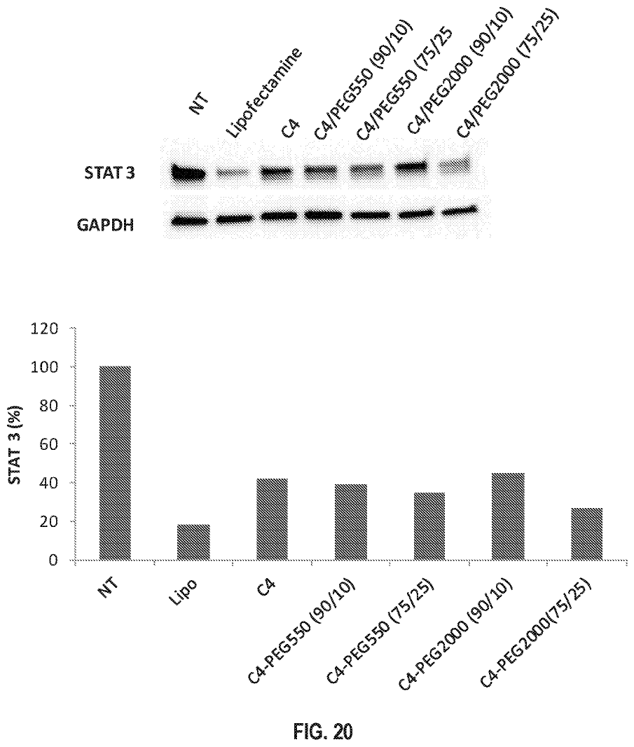

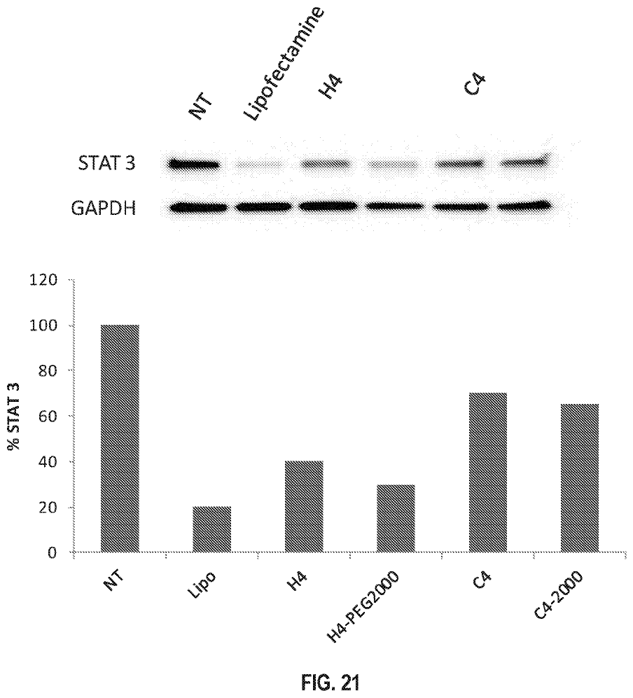

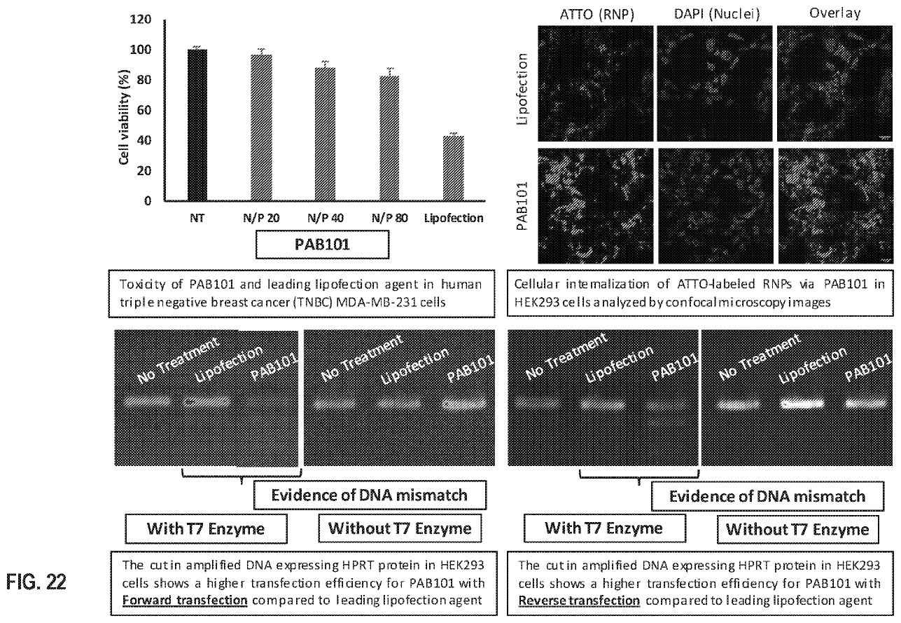

The efficiency of siRNA uptake in this cell line was also confirmed with confocal microscopy images (FIG. 14). The siRNA silencing efficiency was also evaluated in the same cells targeting signal transducer and activator of transcription 3 (STAT3) as a model protein. STAT3 is a well-studied protein with a central role in JAK2/STAT3 pathway and cancer cell proliferation and survival. The expression level of the targeted protein was assessed using Western Blot, which showed comparable silencing efficiency of lead peptides to Lipofectamine™ 2000 (FIGS. 16-21).

Methodology for Cell-Based Assays. Cell Lines: The human breast cancer cell line MDA-MB-231 was purchased from American Type Culture Collection (ATCC; Manassas, VA). MDA-MB-231 was cultured in the DMEM medium. The media was supplemented with 10% (v/v) fetal bovine serum, 100 U/mL penicillin, and 100 μg/ml streptomycin. Cells were maintained in the normal condition of 37° C. and 5% CO₂ under a humidified atmosphere and were sub-cultured when 80-100% confluent.

Internalization of siRNA into Human Cancer Cell Lines: The capability of peptides in internalizing siRNA into human cells was evaluated using FAM-labeled scrambled siRNA and flow cytometry according to the previously reported procedure (Mozaffari S, Bousoik E, Amirrad F, Lamboy R, Coyle M, Hall R, Alasmari A, Mahdipoor P, Parang K, Montazeri Aliabadi H. Amphiphilic Peptides for Efficient siRNA Delivery. Polymers (Basel). 2019; 11(4). doi: 10.3390/polym11040703. PubMed PMID: 30999603; PMCID: PMC6523661). MDA-MB-231 cells were used for these studies and were seeded in 24-well plates (≈200,000 cells per well). After the addition of the siRNA complexes to cell culture media, cells were incubated at 37° C. and standard growth conditions for 24 hours. After the incubation period and for flow cytometry studies, cells were washed with clear HBSS (×2), trypsinized, and fixed using 3.7% formaldehyde solution. Suspended cells were analyzed with a BD-FACSVerse (BD Biosciences; San Jose, CA) using the FITC channel to quantify cell-associated fluorescence. After each flow cytometry analysis, the percentage of cells with fluorescence signal and the mean fluorescence of the cell population were calculated based on the calibration of the signal gated with non-treated cells (as the negative control), so that the auto-fluorescence would be ≈1% of the population.

Gene Silencing. Signal Transducer and Activator of Transcription 3 (STAT3) protein was selected as a model protein to demonstrate the in vitro silencing efficiency of the designed modified peptides. STAT3 has been shown to play a major role in the proliferation and survival of different types of cancer, including breast cancer, and may even be involved in resistance against molecularly targeted drugs (Bousoik “Do Inventors Know Jack” About JAK? A Closer Look at JAK/STAT Signaling Pathway. Front Oncol, 2018, 8:287). Along with Janus Kinase 2 (JAK2), STAT3 is among the major proteins involved in this inter-pathway crosstalk, and latest reports have led to the elucidation of a key role of the JAK/STAT signaling pathway in the development, proliferation, differentiation, and survival of cancer cell. The effect of STAT3 activation on Ras and PI3K/Akt pathways and the connections of JAK2 to PI3K and ERK pathways are examples of these inter-pathway crosstalks.

The siRNA silencing efficiency was also evaluated in the same cells targeting signal transducer and activator of transcription 3 (STAT3) as a model protein STAT3 is a well-studied protein with a central role in JAK2/STAT3 pathway and cancer cell proliferation and survival. The expression level of targeted protein was assessed using Western Blot, which showed comparable silencing efficiency of lead peptides to Lipofectamine™ 2000. Several peptides when used with siRNA, exhibited significant silencing of Stat3 similar to or better than lipofectamine (FIGS. 16-21).

Protein Quantification (Western Blot). The expression of the targeted protein was analyzed by western blot. Cell lysates were prepared according to the standard protocol using RIPA buffer. Briefly, cells exposed to siRNA complexes were collected by trypsinization after 48 h of exposure and were centrifuged at 600-800 RPM for 5 min. The supernatant was discarded, and the cell pellet was washed three times with ice-cold PBS. The pellet was resuspended in RIPA buffer (100 μL of buffer for 25 μL of cell pellet), and the cell lysates were then incubated on ice for one hour, during which the tubes were sonicated for 3 min every 10 min. The tubes were then centrifuged for 15 min at 12,000×g (at 4° C.). Microtubes were pre-cooled to transfer the supernatant, and total protein concentration was determined using BSA assay. Briefly, 200 μL of work reagent (50:1 A:B) was added to 25 μL of standard and unknown samples in triplicate into a 96-well plate, and the plate was mixed on a plate shaker for 30 s. The plate was then incubated at 37° C. and 5% CO₂ for 30 min. The absorbance was measured at 562 nm using SpectraMAX M5 microplate reader. Protein (25 μg) was loaded per well in a 10% Mini-PROTEAN™ TGX Stain-Free™ Protein gel using electrophoresis buffer (0.192 M glycine, 25 mM Tris, 0.1% SDS), and the electrophoresis was run for 60 min with 100 V. The gel was then transferred onto a Trans-Blot™ Turbo™ Mini PVDF membrane (Catalog no. 1704156). Membranes were blocked in BSA 5% for 3 h, and then incubated overnight (at 4° C.) with the primary anti-body (1:1000 in TBS-T). The membrane was then washed with TBS-T three times (5 min each time) and was subsequently incubated with the secondary HRP-linked antibody (1:1000 in TBS-T) for 1 h, followed by the washing steps. Detection was done by ECL Detect Kit using ChemiDoc imager (Bio-Rad).

1-205. (canceled)

206. A compound having the formula:
(R^{z})-(L¹)-(R^{a})ₙ-(L²)-(R^{b})ₙ-(L³)-R^{x},
or a pharmaceutically acceptable salt thereof,
wherein one of R^{a} or R^{b} represents a hydrophobic amino acid residue, and the other represents a charged amino acid residue,
n is in each case selected from 1-10;
L¹, L², and L³ are each independently selected from null or a linker,
R^{z} is null, a lipophilic moiety, a PEG moiety, a lysine amino acid residue, or a cysteine amino acid residue,
R^{x} is NH₂ or forms a bond to R^{z},
wherein said lysine residue may further comprise a lipophilic moiety, a PEG moiety, and/or may form a ring with any of R^{a}, R^{L2}, R^{b} or R^{L3},
wherein said cysteine residues may further comprise a lipophilic moiety, a PEG moiety, and/or may form a ring with any of R^{a}, L², R^{b} or L³;
wherein R^{a} is in each case the same and R^{b} is in each case the same.

207. The compound of claim 206, wherein R^{a} is in each case L-tryptophan.

208. The compound of claim 206, wherein R^{b} is in each case L-arginine.

209. The compound of claim 206, wherein L¹ is a linker having the formula:
—[NH—[CH₂]ₐ—X—[CH₂]_{b}—C(═O)]_{c}—,
wherein a is selected from 0-5;
b is selected from 0-5,
c is selected from 1-10; and
X is null or O.

210. The compound of claim 209, wherein a is 2, X is null, and b is 0.

211. The compound of claim 206, wherein L² is a linker having the formula:
—[NH—[CH₂]ₐ—X—[CH₂]_{b}—C(═O)]_{c}—,
wherein a is selected from 0-5;
b is selected from 0-5, and
c is selected from 1-10;
X is null or O.

212. The compound of claim 211, wherein a is 2, X is null and b is 0.

213. The compound of claim 206, wherein L³ is null.

214. The compound of claim 206, wherein R^{z} is a lipophilic moiety.

215. The compound of claim 214, wherein R^{z} has the formula CH₃(CH₂)_{d}C(═O)—, wherein d is from 5-25.

216. The compound of claim 214, wherein L¹, L², and L³ are each null.

217. The compound of claim 216, wherein R^{x} is NH₂.

218. The compound of claim 206, wherein R^{z} is a cysteine residue having the formula: 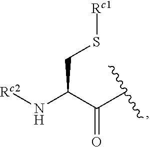
wherein R^{c1} forms a bond to R^{x}; and
R^{c2} is H.

219. The compound of claim 218, having the formula:
[CRRRRRWWWWWC],
wherein the cysteine residues form a disulfide bond.

220. The compound of claim 206, having the formula:
WWWW[CR₅C]WWWW,
wherein the cysteine residues form a disulfide bond.

221. A composition comprising the compound of claim 206, and a nucleic acid.

222. A method of delivering a nucleic acid to a cell, comprising contacting the cell with the composition of claim 221.

223. A method of treating a disease state in a subject in need thereof, comprising administering to the subject the composition of claim 221.

224. A cosmetic composition comprising the compound of claim 206.

225. The composition of claim 221, wherein the composition is a pesticidal or herbical composition.
